SEQUENCE LISTING (41 sequences)

SEQ ID NO: 1 - DNA, 72 bp, Artificial Sequence
  hsa-miR-21 (HGNCMIR21)
ttgtcgggtagcttatcagactgatgttgactgttgaatctcatggcaacaccagtcgatgggctgtctgac

SEQ ID NO: 2 - DNA, 71 bp, Artificial Sequence
  hsa-miR-18a (HGNCMIR18A)
ttgttctaaggtgcatctagtgcagatagtgaagtagattagcatctactgccctaagtgctccttctggc

SEQ ID NO: 3 - DNA, 64 bp, Artificial Sequence
  hsa-miR-375 (HGNCMIR375)
cccgcgacgagcccctcgcacaaaccggacctgagcgttttgttcgttcggctcgcgtgaggca

SEQ ID NO: 4 - DNA, 110 bp, Artificial Sequence
  hsa-miR-181d (HGNCMIR181D)
agtgataatgtagcgagattttctgttgtgcttgatctaaccatgtggttgcgaggtatgagtaaaacatggttccgtcaagcaccatggaacgtcacgcagctttctac

SEQ ID NO: 5 - DNA, 137 bp, Artificial Sequence
  hsa-mir-181d (HGNCMIR181D)
cgtcccctcccctaggccacagccgaggtcacaatcaacattcattgttgtcggtgggttgtgaggactgaggccagacccaccgggggatgaatgtcactgtggctgggccagacacggcttaaggggaatgggga

SEQ ID NO: 6 - DNA, 110 bp, Artificial Sequence
  hsa-mir-181b (HGNCMIR181B1)
ctgtgcagagattattttttaaaaggtcacaatcaacattcattgctgtcggtgggttgaactgtgtggacaagctcactgaacaatgaatgcaactgtggccccgcttt

SEQ ID NO: 7 - DNA, 84 bp, Artificial Sequence
  hsa-mir-491 (HGNCMIR491)
attgacttagctgggtagtggggaacccttccatgaggagtagaacactccttatgcaagattcccttctacctggctgggttg

SEQ ID NO: 8 - DNA, 96 bp, Artificial Sequence
  hsa-mir-455 (HGNCMIR455)
ttccctggcgtgagggtatgtgcctttggactacatcgtggaagccagcaccatgcagtccatgggcatatacacttgcctcaaggcctatgtcat

SEQ ID NO: 9 - DNA, 82 bp, Artificial Sequence
  hsa-mir-130b (HGNCMIR130B)
aggcctgcccgacactctttccctgttgcactactataggccgctgggaagcagtgcaatgatgaaagggcatcggtcaggt

SEQ ID NO: 10 - DNA, 110 bp, Artificial Sequence
  hsa-mir-221 (HGNCMIR221)
gaacatccaggtctggggcatgaacctggcatacaatgtagatttctgtgttcgttaggcaacagctacattgtctgctgggtttcaggctacctggaaacatgttctcc

SEQ ID NO: 11 - DNA, 83 bp, Artificial Sequence
  hsa-mir-193b (HGNCMIR193B)
tgtggtctcagaatcggggttttgagggcgagatgagtttatgttttatccaactggccctcaaagtcccgcttttggggtca

SEQ ID NO: 12 - DNA, 110 bp, Artificial Sequence
  hsa-mir-181a (HGNCMIR181A1)
gagttttgaggttgcttcagtgaacattcaacgctgtcggtgagtttggaattaaaatcaaaaccatcgaccgttgattgtaccctatggctaaccatcatctactccat

SEQ ID NO: 13 - DNA, 71 bp, Artificial Sequence
  hsa-mir-18b (HGNCMIR18B)
gtgttaaggtgcatctagtgcagttagtgaagcagcttagaatctactgccctaaatgccccttctggcac

SEQ ID NO: 14 - RNA, 72 nt, Artificial Sequence
  hsa-miR-21 (HGNCMIR21)
ugucggguagcuuaucagacugauguugacuguugaaucucauggcaacaccagucgaugggcugucugaca

SEQ ID NO: 15 - RNA, 71 nt, Artificial Sequence
  hsa-miR-18a (HGNCMIR18A)
uguucuaaggugcaucuagugcagauagugaaguagauuagcaucuacugcccuaagugcuccuucuggca

SEQ ID NO: 16 - RNA, 64 nt, Artificial Sequence
  hsa-miR-375 (HGNCMIR375)
ccccgcgacgagccccucgcacaaaccggaccugagcguuuuguucguucggcucgcgugaggc

SEQ ID NO: 17 - RNA, 110 nt, Artificial Sequence
  hsa-mir-181d (HGNCMIR181D)
gugauaauguagcgagauuuucuguugugcuugaucuaaccaugugguugcgagguaugaguaaaacaugguuccgucaagcaccauggaacgucacgcagcuuucuaca

SEQ ID NO: 18 - RNA, 137 nt, Artificial Sequence
  hsa-mir-181d (HGNCMIR181D)
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 19 - RNA, 110 nt, Artificial Sequence
  hsa-mir-181b (HGNCMIR181B1)
ccugugcagagauuauuuuuuaaaaggucacaaucaacauucauugcugucgguggguugaacuguguggacaagcucacugaacaaugaaugcaacuguggccccgcuu

SEQ ID NO: 20 - RNA, 84 nt, Artificial Sequence
  hsa-mir-491 (HGNCMIR491)
uugacuuagcuggguaguggggaacccuuccaugaggaguagaacacuccuuaugcaagauucccuucuaccuggcuggguugg

SEQ ID NO: 21 - RNA, 96 nt, Artificial Sequence
  hsa-mir-455 (HGNCMIR455)
ucccuggcgugaggguaugugccuuuggacuacaucguggaagccagcaccaugcaguccaugggcauauacacuugccucaaggccuaugucauc

SEQ ID NO: 22 - RNA, 82 nt, Artificial Sequence
  hsa-mir-130b (HGNCMIR130B)
ggccugcccgacacucuuucccuguugcacuacuauaggccgcugggaagcagugcaaugaugaaagggcaucggucagguc

SEQ ID NO: 23 - RNA, 110 nt, Artificial Sequence
  hsa-mir-221 (HGNCMIR221)
ugaacauccaggucuggggcaugaaccuggcauacaauguagauuucuguguucguuaggcaacagcuacauugucugcuggguuucaggcuaccuggaaacauguucuc

SEQ ID NO: 24 - RNA, 83 nt, Artificial Sequence
  hsa-mir-193b (HGNC193B)
guggucucagaaucgggguuuugagggcgagaugaguuuauguuuuauccaacuggcccucaaagucccgcuuuuggggucau

SEQ ID NO: 25 - RNA, 110 nt, Artificial Sequence
  hsa-mir-181a (HGNC181A1)
ugaguuuugagguugcuucagugaacauucaacgcugucggugaguuuggaauuaaaaucaaaaccaucgaccguugauuguacccuauggcuaaccaucaucuacucca

SEQ ID NO: 26 - RNA, 71 nt, Artificial Sequence
  hsa-mir-18b (HGNCMIR18b)
uguguuaaggugcaucuagugcaguuagugaagcagcuuagaaucuacugcccuaaaugccccuucuggca

SEQ ID NO: 27 - RNA, 22 nt, Artificial Sequence
  hsa-miR-21 (HGNCMIR21)
uagcuuaucagacugauguuga

SEQ ID NO: 28 - RNA, 23 nt, Artificial Sequence
  hsa-miR-18a (HGNCMIR18A)
uaaggugcaucuagugcagauag

SEQ ID NO: 29 - RNA, 22 nt, Artificial Sequence
  hsa-miR-375 (HGNCMIR375)
uuuguucguucggcucgcguga

SEQ ID NO: 30 - RNA, 21 nt, Artificial Sequence
  hsa-mir-181d (HGNCMIR181D)
uugugcuugaucuaaccaugu

SEQ ID NO: 31 - RNA, 23 nt, Artificial Sequence
  hsa-mir-181d (HGNCMIR181D)
aacauucauuguugucggugggu

SEQ ID NO: 32 - RNA, 23 nt, Artificial Sequence
  hsa-mir-181b (HGNCMIR181B)
aacauucauugcugucggugggu

SEQ ID NO: 33 - RNA, 22 nt, Artificial Sequence
  hsa-mir-491 (HGNCMIR491)
aguggggaacccuuccaugagg

SEQ ID NO: 34 - RNA, 22 nt, Artificial Sequence
  hsa-mir-455 (HGNCMIR455)
uaugugccuuuggacuacaucg

SEQ ID NO: 35 - RNA, 22 nt, Artificial Sequence
  hsa-mir-130b (HGNCMIR130B)
cagugcaaugaugaaagggcau

SEQ ID NO: 36 - RNA, 23 nt, Artificial Sequence
  hsa-mir-221 (HGNCMIR221)
agcuacauugucugcuggguuuc

SEQ ID NO: 37 - RNA, 22 nt, Artificial Sequence
  hsa-mir-193b (HGNCMIR193B)
aacuggcccucaaagucccgcu

SEQ ID NO: 38 - RNA, 23 nt, Artificial Sequence
  hsa-mir-181a (HGNCMIR181a)
aacauucaacgcugucggugagu

SEQ ID NO: 39 - RNA, 23 nt, Artificial Sequence
  hsa-mir-18b (HGNCMIR18b)
uaaggugcaucuagugcaguuag

SEQ ID NO: 40 - DNA, 69 bp, Artificial Sequence
  miR-375 construct
tgctgccccgcgacgagcccctcgcacaaaccggacctgagcgttttgttcgttcggctcgcgtgaggc

SEQ ID NO: 41 - DNA, 69 bp, Artificial Sequence
  miR-375 construct
cctggcctcacgcgagccgaacgaacaaaacgctcaggtccggtttgtgcgaggggctcgtcgcggggc

(19) United States
(12) Patent Application Publication
(10) Pub. No.: US 2012/0108655 A1
(43) Pub. Date: May 3, 2012

(54) Methods of Diagnosing and Treating Carcinomas

(75) Inventors: Michele Avissar, Johnston, RI (US); Brock C. Christensen, Hanover, NH (US); Karl T. Kelsey, Brookline, MA (US); Carmen J. Marsit, Grantham, NH (US)

(73) Assignee: BROWN UNIVERSITY, Providence, RI (US)

(21) Appl. No.: 13/148,650

(22) PCT Filed: Mar. 5, 2010
(86) PCT No.: PCT/US10/26388

§ 371 Date: Nov. 7, 2011

Related U.S. Application Data
(60) Provisional application No. 61/209,320, filed on Mar. 5, 2009.

(51) Int. Cl. A61K 31/7088; A61P 35/00; C12Q 1/68; C40B 30/04

(52) U.S. Cl. 514/44 R; 506/9; 435/6.12

(57) ABSTRACT

Methods of diagnosing a carcinoma include comparing the expression of miRNAs in a sample with a control and determining ratios of expression of miRNAs. Methods of treatment include administering a nucleic acid encoding a miR-375 gene product. Methods of optimizing treatment in a subject include determining expression of an miR21-gene product.

### RELATED APPLICATION(S)

This application claims the benefit of U.S. Provisional Application No. 61/209,320, filed on Mar. 5, 2009. The entire teachings of the above application are incorporated herein by reference.

### GOVERNMENT SUPPORT

The invention was supported, in whole or in part, by grants R01CA078609, R01CA100679, and T32ES007272 from the National Institutes of Health, National Cancer Institute and National Institute of Environmental Health Sciences. The Government has certain rights in the invention.

### BACKGROUND OF THE INVENTION

Carcinomas can account for about 80-90% of all cancers in humans. The underlying causes of many types of carcinomas, including head and neck squamous cell carcinoma (HNSCC), are poorly understood. Early detection of carcinomas can be important in determining the course of treatment and in increasing survival rates. Thus, there is a need to develop new, improved and effective methods of diagnosing a carcinoma.

### SUMMARY OF THE INVENTION

The present invention generally relates to methods of diagnosing a carcinoma.

In an embodiment, the invention is a method of diagnosing a carcinoma in a subject, comprising the step of determining at least one expression ratio selected from the group consisting of a miR-21/miR-375 expression ratio, a miR-181d/miR-375 expression ratio, a miR-181b/miR-375 expression ratio, a miR-491/miR-375 expression ratio, a miR-455/miR-375 expression ratio, a miR-18a/miR-375 expression ratio, a miR-130b/miR-375 expression ratio, a miR-221/miR-375 expression ratio, a miR-193b/miR-375 expression ratio, a miR-181a/miR-375 expression ratio, and a miR-18b/miR-375 expression ratio in a sample, wherein a ratio greater than about 1.0 is diagnostic of the carcinoma and identifies a subject that would potentially benefit from a therapy to treat the carcinoma.

In another embodiment, the invention is a method of diagnosing a carcinoma in a subject, comprising the step of comparing an expression level of at least two microRNAs selected from the group consisting of miR-21, miR-181d, miR-181b, miR-491, miR-455, miR-18a, miR-130b, miR-221, miR-193b, miR-181a, miR-18b and miR-375 in a sample from the subject to a corresponding control expression level, wherein a difference in the expression level of the microRNAs in the sample relative to the control expression level is diagnostic of the carcinoma and identifies a subject that would potentially benefit from a therapy to treat the carcinoma.

In a further embodiment, the invention is a method of diagnosing a carcinoma selected from the group consisting of a head squamous cell carcinoma and a neck squamous cell carcinoma in a subject, comprising the step of comparing an expression level of at least one microRNA selected from the group consisting of miR-181d, miR-181b, miR-491, miR-455, miR-18a, miR-130b, miR-221, miR-193b, miR-181a, miR-18b and miR-375 in a sample from the subject to a corresponding control expression level, wherein a difference in the expression level of the microRNA in the sample relative to the control expression level is diagnostic of the head squamous cell carcinoma and the neck squamous cell carcinoma and identifies a subject that would potentially benefit from a therapy to treat the head squamous cell carcinoma and the neck squamous cell carcinoma.

In an additional embodiment, the invention is a method of diagnosing a carcinoma selected from the group consisting of a head squamous cell carcinoma and a neck squamous cell carcinoma in a subject, comprising the step of comparing an expression level of at least one microRNA selected from the group consisting of miR-181d, miR-181b, miR-491, miR-455, miR-18a, miR-130b, miR-221, miR-193b, miR-181a, miR-18b and miR-375 in a sample to a corresponding control expression level, wherein a difference in the expression level of the microRNA in the sample relative to the control expression level is diagnostic of the head squamous cell carcinoma and the neck squamous cell carcinoma and identifies a subject that would potentially benefit from a therapy to treat the head squamous cell carcinoma and the neck squamous cell carcinoma and wherein the sample is not an established cell line.

In another embodiment, the invention is a method of treating a squamous cell carcinoma in a subject, comprising the step of administering a nucleic acid encoding a miR-375 gene product to the subject.

In a further embodiment, the invention is a method of optimizing treatment of a subject having a squamous cell carcinoma, comprising the step of determining an expression level of a miR-21 gene product in a sample from the subject, wherein over expression level of the miR-21 gene product in the sample compared to expression of a reference miR21-gene product identifies a subject that has an aggressive squamous cell carcinoma that would potentially benefit from a therapy to treat the aggressive squamous cell carcinoma.

The methods of the invention can be employed to diagnose a carcinoma in a subject. Advantages of the claimed invention include, for example, improved sensitivity and specificity in discriminating a carcinoma from a normal, non-carcinoma tissue to detect a carcinoma at an early stage.

### BRIEF DESCRIPTION OF THE DRAWINGS

FIG. 1A depicts global normalized signal of six miRNAs (also referred to herein as “miR”) found to be significantly differentially expressed by microarray analysis using the SAM method.

FIG. 1B depicts quantitative real-time PCR analysis showing the relative expression of the same six miRNAs shown in FIG. 2A and confirming differential expression of four of the miRNAs identified as aberrantly expressed in the microarray. * indicates P<0.01.

FIG. 2A depicts validation of differential expression of miR-21 by quantitative realtime PCR in tumor (n=99) and normal (n=14) samples. Boxes denote distribution of expression values from the 25th to 75th percentile. Horizontal lines in boxes represent median values, whiskers represent 5th and 95th percentiles and outliers are denoted by dots. P<0.0001.

FIG. 2B depicts validation of differential expression of miR-375 by quantitative realtime PCR in tumor (n=99) and normal (n=14) samples. Boxes denote distribution of expression values from the 25th to 75th percentile. Horizontal lines in boxes represent median values, whiskers represent 5th and 95th percentiles and outliers are denoted by dots. P<0.0001.

FIG. 2C depicts validation of differential expression of miR-18a by quantitative realtime PCR in tumor (n=99) and normal (n=14) samples. Boxes denote distribution of expression values from the 25th to 75th percentile. Horizontal lines in boxes represent median values, whiskers represent 5th and 95th percentiles and outliers are denoted by dots. P<0.0001.

FIG. 2D depicts validation of differential expression of miR-221 by quantitative realtime PCR in tumor (n=99) and normal (n=14) samples. Boxes denote distribution of expression values from the 25th to 75th percentile. Horizontal lines in boxes represent median values, whiskers represent 5th and 95th percentiles and outliers are denoted by dots. P<0.0001.

FIG. 3A depicts receiver operating curve (ROC) analysis of an expression ratio of miR-221 to miR-375 for differentiation of HNSCC tumors and normal tissues. The curve was constructed using ratio value cut-offs ranging from 0.75 to 1.25 for the ratio. Area under the curve (AUC) value is indicated.

FIG. 3B depicts receiver operating curve (ROC) analysis of an expression ratio of miR-21 to miR-375 for differentiation of HNSCC tumors and normal tissues. The curve was constructed using ratio value cut-offs ranging from 0.75 to 1.25 for the ratio. Area under the curve (AUC) value is indicated.

FIG. 3C depicts receiver operating curve (ROC) analysis of an expression ratio of miR-18a to miR-375 for differentiation of HNSCC tumors and normal tissues. The curve was constructed using ratio value cut-offs ranging from 0.75 to 1.25 for the ratio. Area under the curve (AUC) value is indicated.

FIG. 4A depicts a class prediction analysis using Prediction Analysis of Microarray (PAM) showing misclassification error graphed as a function of the threshold parameter. Threshold was set to include all miRNA predicted by both SAM and ANOVA. Cross-validation was used to calculate misclassification error at each threshold.

FIG. 4B depicts a class prediction analysis using Prediction Analysis of Microarray (PAM) showing cross-validated probabilities for each sample. Threshold was set to include all miRNA predicted by both SAM and ANOVA. Cross-validation was used to calculate misclassification error at each threshold.

FIG. 4C depicts a class prediction analysis using Prediction Analysis of Microarray (PAM) showing tumor and normal scores for each miRNA and confusion matrix showing error associated with class predictions. Threshold was set to include all miRNA predicted by both SAM and ANOVA. Cross-validation was used to calculate misclassification error at each threshold.

FIG. 5 depicts Kaplan-Meier curves showing differences in survival between patients with miR-21 expression in the highest 25th % tile (dotted line) compared to all other patients (solid line). Vertical hatch marks represent censored data.

FIG. 6A depicts precursor miR-375 transfection in FaDu cells. Increase in fold change expression of miR-375 compared to negative control-transfected cells, Expression was normalized to RNU48 expression and was determined as fold-change above negative-control transfected cells by the 2^{-(ΔΔC}_{T}⁾ calculation.

FIG. 6B depicts precursor miR-375 transfection in FaDu cells. Increasing colony formation with increasing concentration of miR-375 precursor.

FIG. 6C depicts precursor miR-375 transfection in FaDu cells. Change in wound healing ability in cells transfected with 5 nM miR-375 precursor compared to control-transfected cells.

FIG. 7A depicts precursor miR-375 transfection in FaDu cells. Increased proliferation rate in miR-375-transfected cells compared to control as measured by proliferation assay.

FIG. 7B depicts precursor miR-375 transfection in FaDu cells. Increased resistance to 0.5 μM CDDP in miR-375-transfected cells compared to negative control as assessed by colony formation assay.

FIG. 8 depicts quantitative real-time PCR showing miR-375 expression following both methods of overexpression. Expression was normalized to RNU48 expression and was determined as fold-change above negative-control transfected cells by the 2^{-(ΔΔC}_{T}⁾ calculation.

FIG. 9A depicts miR-375 expression vector transfection in FaDu cells. Decreased proliferation rate in both miR-375-transfected clones compared to control-transfected cells as measured by proliferation assay.

FIG. 9B depicts miR-375 expression vector transfection in FaDu cells. Increased sensitivity to 0.5 μM CDDP in miR-375-transfected clones compared to negative control as assessed by colony formation assay.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principal features of this invention can be employed in various embodiments without departing from the scope of the invention.

In an embodiment, the invention is a method of diagnosing a carcinoma in a subject, comprising the step of determining at least one expression ratio selected from the group consisting of a miR-21/miR-375 expression ratio, a miR-181d/miR-375 expression ratio, a miR-181b/miR-375 expression ratio, a miR-491/miR-375 expression ratio, a miR-455/miR-375 expression ratio, a miR-18a/miR-375 expression ratio, a miR-130b/miR-375 expression ratio, a miR-221/miR-375 expression ratio, a miR-193b/miR-375 expression ratio, a miR-181a/miR-375 expression ratio, and a miR-18b/miR-375 expression ratio in a sample, wherein a ratio greater than about 1.0 is diagnostic of the carcinoma.

Nucleic acid sequences encoding miRNA gene products and the nucleic acid sequences of premature (also referred to as “mature stem loop”) miRNA gene products and mature miRNA gene products for use in the methods of the invention are known to one of skill in the art. For example, hsa-miR-21 (SEQ ID NO: 27) (HGNC:MIR21) is encoded by SEQ ID NO: 1; hsa-miR-18a (SEQ ID NO: 28) HGNC:MIR18A is encoded by SEQ ID NO: 2; hsa-miR-375 (SEQ ID NO: 29) HGNC:MIR375 is encoded by SEQ ID NO: 3; and hsa-miR-218 (SEQ ID NO: 30) HGNC:218-1 is encoded by SEQ ID NO: 4.

Exemplary miR gene products (SEQ ID NOs: 27-39), nucleic acids encoding the miR gene products (e.g., SEQ ID NOs: 1-13) and mature stem loop miR gene products (also referred to herein as “premature miR gene product”) (SEQ ID NOs: 14-26) can include the following:

hsa-miR-21 (HGNC:MIR21) ref|NT_010783.15|:23192778-23192849
Homo sapiens chromosome 17 genomic contig, GRCh37 reference
primary assembly
(SEQ ID NO: 1)
TTGTCGGGTAGCTTATCAGACTGATGTTGACTGTTGAATCTCATGGCAAC
ACCAGTCGATGGGCTGTCTGAC;
hsa-miR-18a (HGNC:MIR18A) >ref|NT_009952.14|:5092680-5092750
Homo sapiens chromosome 13 genomic contig, GRCh37 reference
primary assembly
(SEQ ID NO: 2)
TTGTTCTAAGGTGCATCTAGTGCAGATAGTGAAGTAGATTAGCATCTACT
GCCCTAAGTGCTCCTTCTGGC;
hsa-miR-375 (HGNC:MIR375) >ref|NT_005403.17|:c70075847-70075784
Homo sapiens chromosome 2 genomic contig, GRCh37 reference
primary assembly
(SEQ ID NO: 3)
CCCGCGACGAGCCCCTCGCACAAACCGGACCTGAGCGTTTTGTTC
GTTCGGCTCGCGTGAGGCA;
hsa-mir-181d (HGNC:MIR181D) ref|NT_011295.11|:5248490-5248626
Homo sapiens chromosome 19 genomic contig, GRCh37 reference
primary assembly
(SEQ ID NO: 4)
AGTGATAATG TAGCGAGATT TTCTGTTGTG CTTGATCTAA
CCATGTGGTT GCGAGGTATG
AGTAAAACATGGTTCCGTCAAGCACCATGG AACGTCACGC
AGCTTTCTAC;
hsa-mir-181d (HGNC:MIR181D) ref|NT_011295.11|:5248490-5248626
Homo sapiens chromosome 19 genomic contig, GRCh37 reference
primary assembly
(SEQ ID NO: 5)
CGTCCCCTCCCCTAGGCCACAGCCGAGGTCACAATCAACATTCATTGTTG
TCGGTGGGTTGTGAGGACTGAGGCCAGACCCACCGGGGGATGAATGTCA
CTGTGGCTGGGCCAGACACGGCTTAAGGGGAATGGGGA;
hsa-mir-181b (HGNC:MIR181B1) ref|NT_004487.19|:c50316752-50316643
Homo sapiens chromosome 1 genomic contig, GRCh37
reference primary assembly
(SEQ ID NO: 6)
CTGTGCAGAGATTATTTTTTAAAAGGTCACAATCAACATTCATTGCTGTC
GGTGGGTTGAACTGTGTGGACAAGCTCACTGAACAATGAATGCAACTGT
GGCCCCGCTTT;
hsa-mir-491 (HGNC:MIR491) GRCh37 reference primary assembly
(SEQ ID NO: 7)
ATTGACTTAGCTGGGTAGTGGGGAACCCTTCCATGAGGAGTAGAACACT
CCTTATGCAAGATTCCCTTCTACCTGGCTGGGTTG;
hsa-mir-455 (HGNC:MIR455) ref|NT_008470.19|:46136245-46136340
Homo sapiens chromosome 9 genomic contig, GRCh37 reference
primary assembly
(SEQ ID NO: 8)
TTCCCTGGCGTGAGGGTATGTGCCTTTGGACTACATCGTGGAAGCCAGCA
CCATGCAGTCCATGGGCATATACACTTGCCTCAAGGCCTATGTCAT;
hsa-mir-130b (HGNC:MIR130B) ref|NT_011520.12|:1398161-1398242
Homo sapiens chromosome 22 genomic contig, GRCh37 reference
primary assembly
(SEQ ID NO: 9)
AGGCCTGCCCGACACTCTTTCCCTGTTGCACTACTATAGGCCGCTGGGAA
GCAGTGCAATGATGAAAGGGCATCGGTCAGGT;
hsa-mir-221 (HGNC:MIR221) ref|NT_079573.4|:c8457437-8457328
Homo sapiens chromosome X genomic contig, GRCh37 reference
primary assembly
(SEQ ID NO: 10)
GAACATCCAGGTCTGGGGCATGAACCTGGCATACAATGTAGATTTCTGT
GTTCGTTAGGCAACAGCTACATTGTCTGCTGGGTTTCAGGCTACCTGGAA
ACATGTTCTCC;
hsa-mir-193b (HGNC:MIR193B) ref|NT_010393.16|:14337823-14337905
Homo sapiens chromosome 16 genomic contig, GRCh37 reference
primary assembly
(SEQ ID NO: 11)
TGTGGTCTCAGAATCGGGGTTTTGAGGGCGAGATGAGTTTATGTTTTATC
CAACTGGCCCTCAAAGTCCCGCTTTTGGGGTCA;
hsa-mir-181a (HGNC:MIR181A1) ref|NT_004487.19|:c50316923-50316814
Homo sapiens chromosome 1 genomic contig, GRCh37 reference
primary assembly
(SEQ ID NO: 12)
GAGTTTTGAGGTTGCTTCAGTGAACATTCAACGCTGTCGGTGAGTTTGGA
ATTAAAATCAAAACCATCGACCGTTGATTGTACCCTATGGCTAACCATCA
TCTACTCCAT;
hsa-mir-18b (HGNC:MIR18B) ref|NT_011786.16|:c17571850-17571780
Homo sapiens chromosome X genomic contig, GRCh37 reference
primary assembly
(SEQ ID NO: 13)
GTGTTAAGGTGCATCTAGTGCAGTTAGTGAAGCAGCTTAGAATCTACTGC
CCTAAATGCCCCTTCTGGCAC;
hsa-miR-21 (HGNC:MIR21) Mature Stem Loop
(SEQ ID NO: 14)
UGUCGGGUAGCUUAUCAGACUGAUGUUGACUGUUGAAUCUCAUGGCA
ACACCAGUCGAUGGGCUGUCUGACA;
hsa-miR-18a (HGNC:MIR18A) Mature Stem Loop
(SEQ ID NO: 15)
UGUUCUAAGGUGCAUCUAGUGCAGAUAGUGAAGUAGAUUAGCAUCUA
CUGCCCUAAGUGCUCCUUCUGGCA;
hsa-miR-375 (HGNC:MIR375) Mature Stem Loop
(SEQ ID NO: 16)
CCCCGCGACGAGCCCCUCGCACAAACCGGACCUGAGCGUUUUGUUCGU
UCGGCUCGCGUGAGGC;
hsa-mir-181d (HGNC:MIR181D) Mature Stem Loop
(SEQ ID NO: 17)
GUGAUAAUGU AGCGAGAUUU UCUGUUGUGC UUGAUCUAAC
CAUGUGGUUG CGAGGUAUGA GUAAAACAUG GUUCCGUCAA
GCACCAUGGA ACGUCACGCA GCUUUCUACA;
hsa-mir-181d (HGNC:MIR181D) Mature Stem Loop
(SEQ ID NO: 18)
GUCCCCUCCCCUAGGCCACAGCCGAGGUCACAAUCAACAUUCAUUGUU
GUCGGUGGGUUGUGAGGACUGAGGCCAGACCCACCGGGGGAUGAAUG
UCACUGUGGCUGGGCCAGACACGGCUUAAGGGGAAUGGGGAC;
hsa-mir-181b (HGNC:MIR181B1) Mature Stem Loop
(SEQ ID NO: 19)
CCUGUGCAGAGAUUAUUUUUUAAAAGGUCACAAUCAACAUUCAUUGC
UGUCGGUGGGUUGAACUGUGUGGACAAGCUCACUGAACAAUGAAUGC
AACUGUGGCCCCGCUU;
hsa-mir-491 (HGNC:MIR491) Mature Stem Loop
(SEQ ID NO: 20)
UUGACUUAGCUGGGUAGUGGGGAACCCUUCCAUGAGGAGUAGAACAC
UCCUUAUGCAAGAUUCCCUUCUACCUGGCUGGGUUGG;
hsa-mir-455 (HGNC:MIR455) Mature Stem Loop
(SEQ ID NO: 21)
UCCCUGGCGUGAGGGUAUGUGCCUUUGGACUACAUCGUGGAAGCCAGC
ACCAUGCAGUCCAUGGGCAUAUACACUUGCCUCAAGGCCUAUGUCAUC;
hsa-mir-130b (HGNC:MIR130B) Mature Stem Loop
(SEQ ID NO: 22)
GGCCUGCCCGACACUCUUUCCCUGUUGCACUACUAUAGGCCGCUGGGA
AGCAGUGCAAUGAUGAAAGGGCAUCGGUCAGGUC;
hsa-mir-221 (HGNC:MIR221) Mature Stem Loop
(SEQ ID NO: 23)
UGAACAUCCAGGUCUGGGGCAUGAACCUGGCAUACAAUGUAGAUUUC
UGUGUUCGUUAGGCAACAGCUACAUUGUCUGCUGGGUUUCAGGCUAC
CUGGAAACAUGUUCUC;
hsa-mir-193b (HGNC:MIR193B) Mature Stem Loop
(SEQ ID NO: 24)
GUGGUCUCAGAAUCGGGGUUUUGAGGGCGAGAUGAGUUUAUGUUUUA
UCCAACUGGCCCUCAAAGUCCCGCUUUUGGGGUCAU;
hsa-mir-181a (HGNC:MIR181A1) Mature Stem Loop
(SEQ ID NO: 25)
UGAGUUUUGAGGUUGCUUCAGUGAACAUUCAACGCUGUCGGUGAGUU
UGGAAUUAAAAUCAAAACCAUCGACCGUUGAUUGUACCCUAUGGCUA
ACCAUCAUCUACUCCA;
hsa-mir-18b (HGNC:MIR18B) Mature Stem Loop
(SEQ ID NO: 26)
UGUGUUAAGGUGCAUCUAGUGCAGUUAGUGAAGCAGCUUAGAAUCUA
CUGCCCUAAAUGCCCCUUCUGGCA;
hsa-miR-21 (HGNC:MIR21)
(SEQ ID NO: 27)
UAGCUUAUCAGACUGAUGUUGA;
hsa-miR-18a (HGNC:MIR18A)
(SEQ ID NO: 28)
UAAGGUGCAUCUAGUGCAGAUAG;
hsa-miR-375 (HGNC:MIR375)
(SEQ ID NO: 29)
UUUGUUCGUUCGGCUCGCGUGA;
hsa-mir-181d (HGNC:MIR181D)
(SEQ ID NO: 30)
UUGUGCUUGAUCUAACCAUGU;
hsa-mir-181d (HGNC:MIR181D)
(SEQ ID NO: 31)
AACAUUCAUUGUUGUCGGUGGGU;
hsa-mir-181b (HGNC:MIR181B1)
(SEQ ID NO: 32)
AACAUUCAUUGCUGUCGGUGGGU;
hsa-mir-491 (HGNC:MIR491)
(SEQ ID NO: 33)
AGUGGGGAACCCUUCCAUGAGG;
hsa-mir-455 (HGNC:MIR455)
(SEQ ID NO: 34)
UAUGUGCCUUUGGACUACAUCG;
hsa-mir-130b (HGNC:MIR130B)
(SEQ ID NO: 35)
CAGUGCAAUGAUGAAAGGGCAU;
hsa-mir-221 (HGNC:MIR221)
(SEQ ID NO: 36)
AGCUACAUUGUCUGCUGGGUUUC;
hsa-mir-193b (HGNC:MIR193B)
(SEQ ID NO: 37)
AACUGGCCCUCAAAGUCCCGCU;
hsa-mir-181a (HGNC:MIR181A1)
(SEQ ID NO: 38)
AACAUUCAACGCUGUCGGUGAGU;.
hsa-mir-18b (HGNC: MIR18B)
(SEQ ID NO: 39)
UAAGGUGCAUCUAGUGCAGUUAG.

“Expression ratio,” as used herein, refers to the value of one microRNA (miR) relative to another miR. For example, a miR-21/miR-375 expression ratio is a value obtained when an expression level of miR-21 is divided by an expression level of miR-375.

Exemplary techniques for determining microRNA expression are well known in the art and include, for example, microarray-based methods, reverse-transcriptase polymerase chain reaction (RT-PCR) (e.g., quantitative RT-PCR), Northern-blot analysis and in situ hybridization.

The subject diagnosed by the methods described herein can be a human subject or a non-human subject (e.g., monkey, rat, mouse).

In an embodiment, the carcinoma diagnosed by the methods of the invention includes a carcinoma of non-glandular origin, such as at least one member selected from the group consisting of a squamous cell carcinoma, a basal cell carcinoma, a transitional cell carcinoma, and an undifferentiated carcinoma. Exemplary squamous cell carcinomas include at least one member selected from the group consisting of a head squamous cell carcinoma, a neck squamous cell carcinoma, a skin squamous cell carcinoma, a prostate squamous cell carcinoma, a lung squamous cell carcinoma, a vaginal squamous cell carcinoma and a cervical squamous cell carcinoma.

Exemplary head squamous cell carcinomas that can be diagnosed by the methods described herein can include at least one member selected from the group consisting of an oral cavity squamous cell carcinoma (e.g., tongue squamous cell carcinoma, squamous cell carcinoma of floor of mouth, squamous cell carcinoma of the wall of mouth, gingivae squamous cell carcinoma, hard palate squamous cell carcinoma, soft palate squamous cell carcinoma), a nasal cavity squamous cell carcinoma, a carcinoma of the paranasal sinuses and a nasopharyngeal squamous cell carcinoma. The oral cavity squamous cell carcinoma can include at least one member selected from the group consisting of a tongue squamous cell carcinoma, a squamous cell carcinoma of floor of mouth, a squamous cell carcinoma of the wall of mouth, a gingivae squamous cell carcinoma, a hard palate squamous cell carcinoma and a soft palate squamous cell carcinoma.

The squamous cell carcinoma can be a neck squamous cell carcinoma. Exemplary neck squamous cell carcinomas include a pharyngeal squamous cell carcinoma (e.g., an oropharyngeal squamous cell carcinoma, a hypopharyngeal squamous cell carcinoma), a laryngeal squamous cell carcinoma and a tracheal squamous cell carcinoma. The pharyngeal squamous cell carcinoma can include at least one member selected from the group consisting of an oropharyngeal squamous cell carcinoma and a hypopharyngeal squamous cell carcinoma.

The carcinoma diagnosed by the methods of the invention can also be at least one member selected from the group consisting of an adenocarcinoma and a carcinoma that includes cells of glandular and non-glandular origin, such as an adenosquamous carcinoma).

Exemplary adenocarcinomas that can be diagnosed by the methods of the invention can include at least one member selected from the group consisting of an adenocarcinoma of the colon, an adenocarcinoma of the lung, an adenocarcinoma of the ovary, an adenocarcinoma of the breast, an adenocarcinoma of the pancreas, an adenocarcinoma of the prostate, an adenocarcinoma of the stomach, an adenocarcinoma of the urachus, an adenocarcinoma of the vagina, an adenocarcinoma not otherwise specified (NOS), a cholangiocarcinoma, an adenoid cystic carcinoma, a hepatocellular carcinoma, a renal cell carcinoma, an adrenocorticol carcinoma and an esophageal adenocarcinoma.

The sample employed in the methods described herein can include at least one member selected from the group consisting of a cell sample, a tissue sample and a fluid sample. The sample can be from a subject (e.g., a biopsy, a swab, a saliva sample, a sputum sample, a mouth rinse, a blood serum sample, a blood plasma sample). Alternatively, the sample can be a cell line or cultured cell sample, such as a cell line and a cultured cell sample prepared from a sample from a subject.

Exemplary tissue samples for use in the methods of the invention include at least one member selected from the group consisting of an oral cavity sample, a laryngeal tissue sample, an esophageal tissue sample, a uvula tissue sample, a skin tissue sample, a lip tissue sample, a rectal tissue sample, a renal tissue sample, a bladder tissue sample, a prostate tissue sample, a lung tissue sample and a cervical tissue sample.

The tissue sample employed in the methods of the invention can include at least a portion of a tumor. “At least a portion,” as used herein in reference to a sample, means any part or the entirety of a sample. The tumor can be a malignant tumor, a pre-malignant tumor or a benign tumor. The tumor can be a primary tumor or a metastatic tumor. The tumor can be of any stage, for example a Stage 0 tumor, a Stage I tumor, a Stage II tumor, a Stage III tumor, or a Stage IV tumor, according to an appropriate staging system (e.g., the TNM Classification of Malignant Tumors Staging System). The type and class of tumor, and tumor stage, can be readily determined by one of ordinary skill in the art.

The sample employed in the methods of the invention can be obtained from the subject.

In an embodiment, the tumor is an early-stage tumor. An “early-stage tumor,” as used herein, refers to a tumor that includes at least one member selected from the group consisting of a Stage 0 tumor, a Stage I tumor and a Stage II tumor. An early stage tumor can be classified based on, for example, the TNM Classification of Malignant Tumors Staging System.

Human papilloma virus (HPV) infection is a major risk factor for certain types of carcinoma, such as head and neck squamous cell carcinoma). In an embodiment, the tissue sample includes at least a portion of an HPV-positive tumor. In another embodiment, the tissue sample includes at least a portion of an HPV-negative tumor.

The tissue sample employed in the methods of the invention can include at least a portion of a preneoplastic lesion. Preneoplastic lesions diagnosed by the methods of the invention can include at least one member selected from the group consisting of an actinic keratosis, an atypical adenomatous hyperplasia, a cutaneous horn, a squamous cell carcinoma in situ, a keratoacanthoma, an oral leukoplakia and a pharyngeal leukoplakia.

In another embodiment, the invention is a method of diagnosing a carcinoma in a subject, comprising the step of comparing an expression level of at least two microRNAs selected from the group consisting of miR-21, miR-181d, miR-181b, miR-491, miR-455, miR-18a, miR-130b, miR-221, miR-193b, miR-181a, miR-18b and miR-375 in a sample from the subject to a corresponding control expression level, wherein a difference in the expression level of the microRNAs in the sample relative to the control expression level is diagnostic of the carcinoma and identifies a subject that would potentially benefit from a therapy to treat the carcinoma.

An expression level of at least two microRNAs that are over-expressed in a carcinoma (e.g., miR-21, miR-181d, miR-181b, miR-491, miR-455, miR-18a, miR-130b, miR-221, miR-193b, miR-181a, and miR-18b) can be compared to a microRNA that is underexpressed in the carcinoma (e.g., miR-375) to thereby diagnose the carcinoma. The comparison can be a relative comparison, such as comparing the levels of miRNA that are over and underexpressed, or the comparison can be an expression ratio.

Expression levels of microRNAs can be readily determined by quantitative methods as described herein, such as nucleic acid amplification assays. The methods described herein can identify over-expression (increases) or under-expression (decreases) of microRNAs (miRNAs) compared to a control or a reference miRNA. Over-expression or under-expression can be correlated with subject characteristics (e.g., age, risk factors, such as alcohol consumption and smoking) and carcinoma characteristics (e.g., grade, stage, aggressive, less aggressive, invasive).

Over and under expression of genes described herein can be assessed by determining the Hazard Ratio (HR) by the methods described herein, The Hazard Ration is derived from a survival analysis and describes the effect of an explanatory variable on the risk of an event. It is similar in concept to an Odds Ratio, except it is based on time-dependent data. A Hazard Ratio is calculated from a Cox Proportional Hazards Model that allows adjustment for potential confounders in the analysis of an association between some variable (e.g., miRNA expression) and risk of death. A Hazard Ratio greater than one (1) suggests an increased risk of death or morbidity, while a Hazard Ratio less than one (1) suggests a decreased risk of death (e.g., a protective factor). For example, an HR of 1.68 for high miR-21 expression is interpreted to mean that if a subject has head and neck squamous cell carcinoma and high miR-21 expression, the subject is 68% more likely to die at any given time, compared to a person with head and neck squamous cell carcinoma and low miR-21 expression, controlled for age, gender and tumor stage. When those factors are controlled, and the miR expression is still significant, the miR expression is considered to be an independent predictor (e.g., independent of other known risk factors) of patient survival.

In yet another embodiment, the invention is a method of diagnosing a carcinoma in a subject, comprising the step of comparing an expression level of at least two microRNAs selected from the group consisting of miR-21, miR-181d, miR-181b, miR-491, miR-455, miR-18a, miR-130b, miR-221, miR-193b, miR-181a, miR-18b and miR-375 in a sample to a corresponding control expression level, wherein a difference in the expression level of the microRNAs in the sample relative to the control expression level is diagnostic of the carcinoma and wherein the sample is not an established cell line.

“An established cell line,” as used herein, refers to a cell line that is commercially available. The established cell line is a cell line that has the ability to proliferate indefinitely consequent to a mutation. For example, established cell lines can include a FaDu established cell line (hypopharnyngeal carcinoma), HN6 established cell line (base of tongue primary carcinoma), HN13 established cell line (tongue primary carcinoma), UM-SCC9 established cell line (tongue primary carcinoma), UMSCC47 established cell line (tongue primary carcinoma), UM-SCC10A established cell line (larynx primary carcinoma), UM-SCC11A established cell line (larynx primary carcinoma), UM-SCC38 established cell line (tonsil primary carcinoma), UMSCC4 established cell line (tonsil primary carcinoma), JHU-011 established cell line (laryngeal carcinoma), JHU-012 established cell line (neck node metastasis), JHU-019 established cell line (base of tongue carcinoma) and OKF6 established cell line (oral keratinocyte carcinoma). Cell lines that are obtained or immortalized from samples obtained from the subjects that are diagnosed by the methods of the invention are not established cell lines.

“Corresponding control expression level,” as used herein, refers to an expression level of a microRNA observed in a non-carcinoma sample of the same microRNA who expression level is being evaluated in the sample from the subject. For example, if the expression level of miR-181d is being evaluated in the subject the corresponding miR-181d would be a control or normal sample of miR181d. The corresponding control expression level can be an expression level of a microRNA in a non-carcinoma sample (e.g., a non-carcinoma sample from the same subject, a non-carcinoma sample from a different subject). Alternatively, the corresponding control expression level can be a reference standard for a typical expression level of a microRNA in a non-carcinoma sample.

A difference in the expression level of the miRNA in the sample compared to the corresponding control sample is any difference in the level of a microRNA in a sample from a subject relative to a corresponding control expression level. For example, if the level of microRNA in the sample from the subject is different (greater or less) than the corresponding control level, the subject has a diagnosis of a high probability of having a carcinoma (i.e., a positive prediction that the subject has a carcinoma). If the level of one or more selected microRNAs in the sample from the subject is identical to, or essentially the same as, the corresponding control level, it is unlikely that the subject has a carcinoma (i.e., a negative prediction that the subject has a carcinoma).

The expression level of the microRNA in the sample from the subject can be diagnostic of a carcinoma when it is either greater or less than a corresponding control expression level. For example, in an embodiment, the expression level of a particular microRNA in the sample from the subject can be diagnostic of a carcinoma when it is at least about 2-fold, at least about 2.5-fold, at least about 3-fold, at least about 3.5-fold, at least about 4-fold, at least about 4.5-fold, or at least about 5-fold greater than a corresponding control expression level.

The expression level of miR-18a can be determined in a sample. When the expression level of miR-18a in the sample is greater (e.g., at least about 2.5-fold greater) than a corresponding control, miR-18a expression level is predictive of a carcinoma in the subject.

The expression level of miR-21 can be determined in a sample. When the expression level of miR-21 is greater (e.g., at least about 3.5-fold greater) than a corresponding control, miR-21 expression level is predictive of a carcinoma in the subject.

The expression level of miR-221 can be determined in a sample. When the expression level of miR-221 is greater (e.g., at least about 2-fold greater) than a corresponding control, miR-21 expression level is predictive of a carcinoma in the subject.

In another embodiment, the expression level of miR-375 can be determined in a sample. When the expression level of miR-375 is less (e.g., at least about 20-fold less) than a corresponding control, miR-375 expression level is predictive of a carcinoma in the subject.

In a further embodiment, the invention is a method of diagnosing a carcinoma selected from the group consisting of a head squamous cell carcinoma and a neck squamous cell carcinoma in a subject, comprising the step of comparing an expression level of at least one microRNA selected from the group consisting of miR-181d, miR-181b, miR-491, miR-455, miR-18a, miR-130b, miR-221, miR-193b, miR-181a, miR-18b and miR-375 in a sample from the subject to a corresponding control expression level, wherein a difference in the expression level of the microRNA in the sample relative to the control expression level is diagnostic of the head squamous cell carcinoma or the neck squamous cell carcinoma and identifies a subject that would potentially benefit from a therapy to treat the head squamous cell carcinoma and the neck squamous cell carcinoma.

An expression level of at least one microRNAs that is over-expressed in a carcinoma (e.g., miR-181d, miR-181b, miR-491, miR-455, miR-18a, miR-130b, miR-221, miR-193b, miR-181a, and miR-18b) can be compared to a microRNA that is underexpressed in the carcinoma (e.g., miR-375) to thereby diagnose the carcinoma. The comparison can be a relative comparison, such as comparing the levels of miRNA that are over and underexpressed, or the comparison can be an expression ratio.

The method of diagnosing a carcinoma selected from the group consisting of a head squamous cell carcinoma and a neck squamous cell carcinoma in a subject by comparing an expression level of at least one microRNA selected from the group consisting of miR-181d, miR-181 b, miR-491, miR-455, miR-18a, miR-130b, miR-221, miR-193b, miR-181a, miR-18b and miR-375 in a sample from the subject to a corresponding control expression level, wherein a difference in the expression level of the microRNA in the sample relative to the control expression level is diagnostic of the head squamous cell carcinoma or the neck squamous cell carcinoma can further include the step of comparing the expression level of miR-21 in the sample from the subject to a corresponding miR-21 control expression level.

In still another embodiment, the invention is a method of treating a squamous cell carcinoma in a subject, comprising the step of administering a nucleic acid encoding a miR-375 gene product to the subject. The squamous cell carcinoma treated by the methods of the invention can be at least one member selected from the group consisting of a head squamous cell carcinoma and a neck squamous cell carcinoma. The nucleic acid encoding the miR-375 gene product can include an expression vector. The expression vector can include a promoter, such as at least one member selected from the group consisting of an RNA polymerase II promoter (e.g., a cytomegalovirus (CMV) promoter, an elongation factor 1 (EF-1) promoter, an hPGK promoter) and an RNA polymerase III promoter (e.g., a U6 promoter, an H1 promoter). The RNA polymerase II promoter can include a constitutive promoter. The constitutive promoter can include a human cytomegalovirus immediate early promoter. The nucleic acid encoding the miR-375 gene product can include a nucleic acid that encodes a premature miR-375 gene product. The expression vector can include at least one member selected from the group consisting of a bacterial vector and a lentiviral vector. Suitable expression vectors for use in the methods of the invention include, for example, the BLOCK-iT™ Pol II miR RNAi vector (Invitrogen) and BLOCK-iT™ shRNA vectors for RNAi (Invitrogen), the miRNASelect™ pEP mir Cloning and Expression Vector (Cell Biolabs, Inc.), pLKO.1 vectors (Sigma-Aldrich), BLOCK-iT™ Lentiviral Pol II or III miR RNAi Expression System (Invitrogen) and miR-express Human Lentiviral microRNA Vectors (Thermo Fisher).

Exemplary vectors and promoters can include the following:

BLOCK-iT™ Pol II miR RNAi vector can be used with at least one member selected from the group consisting of an RNA Polymerase II expression system (RNA Pol II promoters are promoters used to transcribe most protein coding genes of a mammalian cell), an EF-1a and an alpha-1-anti-trypsin promoters (normal mammalian gene promoters). In particular, CMV (cytomegalovirus) immediate early Pol II promoter can be employed to drive transcription.

BLOCK-iT™ shRNA vectors for RNAi can employ an RNA Polymerase Type III promoter for expression (Pol III promoters are promoter which drive transcription of many constitutive non-coding RNAs in the cell, like ribosomal RNAs), such as the U6 promoter or the H1 promoter.

miRNASelect™ pEP mir Cloning and Expression Vector can employ a Pol II promoter, such as an EF-1 promoter.

pLKO.1 vectors can employ Pol III (U6) and Pol II (CMV, hPGK) promoters.

BLOCK-iT™ Lentiviral Pol II or III miR RNAi Expression System can employ a Pol II promoter (e.g., CMV, EF-1) or Pol III (e.g., U6) and inserts the promoter and miR construct into lentiviral production vector to generate replication-incompetent Lentivirus that can transduce dividing and non-dividing mammalian cells.

miR-express Human Lentiviral microRNA Vectors can employ an RNA Pol II CMV promoter.

The use of siRNA as therapeutics agents is growing in interest, and similarly growing are methods for delivery of therapeutic siRNAs to appropriate target tissues and organs. MicroRNAs, which share functional similarity to siRNA, but are produced from an initial hairpin structure, may also be suitable as therapeutic agents. The miR-375 gene product can be employed as a therapeutic agent in human cancer cell lines, to determine the utility of overexpression of this miRNA on growth of the cells, and their response to chemotherapeutic agents.

Two different systems were used for overexpression of miR-375 in the invasive HNSCC cell line FaDu. The first system involves transient transfection with a synthetic double-stranded RNA molecule designed to mimic the mature miRNA (Pre-miR™ miRNA Precursor Molecules from Ambion). Functioning like small interfering RNAs (siRNAs), these miRNA precursors are chemically modified to ensure that the appropriate strand becomes incorporated into the RISC. A Pre-miR™ of miR-375 was commercially available and purchased. This system has been used in-vitro and in animal models as a method for delivery of an siRNA. In the case of animal delivery, the synthetic oligos would be administered systemically and taken up by the cells, wherein they can act on their target mRNA. Studies such as Song et al (Nature Medicine 9, 347-351 (2003)) demonstrated that injection of synthetic siRNA duplexes in the tail vein of mice led to their uptake by the liver, although the uptake by other tissues was not reported.

The second method utilizes an expression vector-based system to allow expression of an engineered miRNA sequence from a Pol II promoter (Block-iT™ Pol II miR RNAi Expression Vector from Invitrogen). This expression vector contains the human cytomegalovirus (CMV) immediate early promoter, allowing for constitutive miRNA expression in mammalian cells. Double stranded oligos which were inserted into a vector to produce the mature miRNA within the cells. This type of vector could also be systemically administered, but numerous studies have shown poor uptake of this type of vector. This vector, though, utilizes Invitrogen's Gateway®-adapted expression vector, which is designed to allow for recombination into other tissue-specific, regulated, or lentiviral vector systems. Thus, this vector can be recombined to allow for specific viral delivery of the expression plasmid to target cells, which could be a more specific and efficacious method of therapeutic delivery.

In a prefer embodiment, the invention is a method of optimizing treatment of a subject having a squamous cell carcinoma (e.g., at least one member selected from the group consisting of a head squamous cell carcinoma and a neck squamous cell carcinoma), comprising the step of determining an expression level of a miR-21 gene product in a sample from the subject, wherein the overexpression level of the miR-21 gene product in the sample compared to expression in a reference miR2′-gene product identifies a subject that has an aggressive squamous cell carcinoma that would potentially benefit from a therapy to treat the aggressive squamous cell carcinoma.

“Reference,” as used herein with respect to expression of a miR gene product, such as miR-21, refers to expression of an miR gene product in a sample obtained from a subject that has a carcinoma with a relatively favorable prognosis.

“Aggressive,” as used herein with respect to a squamous cell carcinoma, refers to a carcinoma that is associated with an increased morbidity. As described herein, miRNA expression can be predictive of an aggressive carcinoma, which may or may not correlate with the stage (I, II, III or IV) of the carcinoma. For example, miRNA expression in a sample of a carcinoma (e.g., squamous cell carcinoma such as a head and neck squamous cell carcinoma) that is greater than about 2.0 fold to about 4.0 fold (e.g., about 2.5 fold, about 2.6 fold, about 3.0 fold, about 3.5 fold) of that observed in a control (e.g., normal or noncancerous sample) reference (e.g., sample from a carcinoma that is known not be aggressive) sample can indicate a more aggressive carcinoma. In general, early stage disease (non-metastatic disease) is treated with wide surgical excision or curative radiation therapy alone or in combination. In combination, radiation therapy is often used first in order to reduce the size of the tumor in hopes of improving the cosmetic and functional results from surgery. Chemotherapy is generally used in organ preservation protocols for laryngeal and hypopharyngeal tumors, and often plays a role in palliative care of recurrent disease. Identification of primary tumors that will respond to curative radiation therapy (likely considered the lesser aggressive of the therapy regimens) would be ideal, as this could avoid often disfiguring and function-limiting surgeries to the head and neck. Chemotherapeutic regimens are also under investigation for this disease, including front-line therapies, as targeted therapeutics, and so identifying patients who may benefit from such strategies would be an advantage. Exemplary therapies to treat aggressive squamous cell carcinomas could include, for example, a combination of surgery, post-surgical radiation and chemotherapy. Therapies to treat non-aggressive carcinomas generally employ a single (e.g., radiation therapy alone, surgery alone) therapy and generally do not include chemotherapy.

“Therapeutically effective,” as used herein refers to an amount of an miRNA gene product, nucleic acid encoding an miRNA gene product, chemotherapeutic agent or other suitable therapy, such as radiation therapy that can produce a measurable positive effect in a subject, such as a regression in a carcinoma.

### EXEMPLIFICATION

### Example 1

Head and neck squamous cell carcinoma includes carcinomas arising from the epithelium of the oral cavity, pharynx, and larynx, and is the sixth most common malignancy worldwide (1). The major risk factors for the disease are tobacco and alcohol use, and human papillomavirus (HPV) infection (2-4). Despite advances in detection, as well as surgical and chemotherapeutic treatments over recent decades, the five year survival rate for head and neck squamous cell carcinoma has remained around 50%, one of the lowest of the major cancers (5). Frequent late stage diagnosis, formation of additional primary tumors and regional and distant metastases all contribute to this poor survival rate (2).

A better understanding of the molecular pathways that give rise to head and neck squamous cell carcinoma is essential in the identification of novel molecular biomarkers that have clinical utility in predicting prognosis and therapeutic efficacy, as well as in designing targeted therapy for this disease. In recent years, gene expression profiling technologies have become increasingly sophisticated, allowing investigators to explore their diagnostic and therapeutic potential as biomarkers in cancers (6-8). These biomarkers, however, have had limited success in the clinical setting and, to date, limited utility in further elucidating mechanisms of head and neck squamous cell carcinoma carcinogenesis.

The discovery of microRNAs (miRNAs), about 22 nucleotide long, non-coding RNA molecules, has revolutionized our understanding of the modulation of gene expression. Nearly 700 miRNAs have been identified in humans, a number that is rapidly growing and expected to reach 1,000 or higher (9). Highly ubiquitous and largely conserved across species, miRNAs regulate gene expression post-transcriptionally by base-pairing, usually imperfectly, to the 3′-untranslated region (10) of a cognate messenger RNA (11). The interaction of a miRNA with a target mRNA transcript results either in translational repression of the mRNA or in its direct degradation (11). Due to the partial complementarity between miRNAs and their target transcripts, a single miRNA is capable of simultaneously regulating up to hundreds of genes, giving rise to an enormous modulatory potential (12).

Through their targets, miRNAs are known to play important roles in cell differentiation, proliferation, and apoptosis (13). Different cancer types have been associated with miRNA expression profiles that vary between the tumor tissues and the corresponding normal tissue (19-21). Moreover, some studies have identified miRNA expression profiles that can distinguish different tumor subtypes or developmental lineages, which may have clinical applications in diagnostics and tumor staging (16, 22).

As described herein, the expression of miRNAs in normal head and neck epithelia were compared with primary head and neck squamous cell carcinoma tumors and cultured head and neck squamous cell carcinoma cell lines. Differences in expression are described herein, which may be important in differentiating disease and as markers that diagnosis head and neck squamous cell carcinoma. Upon identifying miRNAs specifically altered in head and neck cancer, a subset of these miRNAs were validated in a larger population of tumors to identify a clinically-applicable diagnostic tool.

The data described herein shows that a microRNA expression ratio can distinguish between non-diseased tissue and tumor tissue with great accuracy in the context of head and neck squamous cell carcinoma, an important public health concern worldwide. The ratio of miR-221:miR-375 showed high discriminatory potential, with a sensitivity of about 92% and specificity of about 93% in distinguishing tumor from normal tissue, which may be a simple molecular marker for diagnosing head and neck squamous cell carcinoma.

### Purpose:

miRNAs altered in head and neck squamous cell carcinoma were identified to determine whether miRNA expression is predictive of disease.

### Experimental Design:

RNA was isolated from fresh frozen primary tumors, fresh frozen non-diseased head and neck epithelial tissues, and head and neck squamous cell carcinoma cell lines, and profiled for the expression of 662 miRNAs by microarray. The miRNAs that were both differentially expressed on the array and by qRT-PCR were subsequently validated by qRT-PCR using a total of 99 head and neck squamous cell carcinoma samples and 14 normal epithelia.

### Results:

A marked difference in miRNA expression pattern was observed between tumors and cell lines. Eighteen miRNAs were significantly altered in their expression between normal tissues and tumors. Four of these miRNAs were validated in the larger sample series, and each showed significant differential expression (P<0.0001). Further, an expression ratio of miR-221:miR-375 demonstrated a high sensitivity (0.92) and specificity (0.93) for disease prediction.

### Conclusions:

These data suggest that cultured tumor cell lines are inappropriate for miRNA biomarker identification, and that the pattern of miRNA expression in primary head and neck tissues is reflective of disease status, with certain miRNAs exhibiting strong predictive potential. These results show that miR-221 and miR-375 may be important diagnostic biomarkers.

### Materials and Methods

head and neck squamous cell carcinoma samples and cell lines.

Non-diseased head and neck epithelial tissue were obtained from the National Disease Research Interchange and consisted of fresh-frozen tongue, larynx and uvula samples. All fresh-frozen head and neck squamous cell carcinoma samples were obtained, with informed consent after Institutional Review Board approval at participating hospitals, as part of a population-based case-control study of head and neck squamous cell carcinoma spanning December 1999 to December 2003 in the Greater Boston Metropolitan area. The fresh-frozen tumors originated from uvula, larynx, floor of mouth, and tongue resections. Details of this study have been described previously (23). Study pathologists confirmed greater than about 75% tumor in all head and neck squamous cell carcinoma samples tested. FaDu and Cal27, head and neck squamous cell carcinoma cell lines, were obtained from American Type Culture Collection (ATCC) and maintained in Eagle's Minimum Essential Medium and Dulbecco's Modified Eagle's Medium, respectively, both supplemented with fetal bovine serum to a final concentration of 10%.

### RNA Isolation and Microarray Profiling.

Total RNA was isolated from normal tissues, tumors, and cell lines using the mirVANA™ RNA Isolation Kit (Ambion, Inc., Austin, Tex.) according to the manufacturer's protocol. RNA was quantified using the NanoDrop™ ND-1000 spectrophotometer (Nanodrop, Wilmington, Del.), aliquoted, and stored at −80° C. briefly until needed. Total RNA (about 5 μg) was evaluated for miRNA profiling studies at Asuragen Services using the mirVANA™ miRNA Bioarrays platform v2 (Ambion, Inc., Catalog No. AM1566V2) as single channel format according to the standard operating procedures of the company, including pre-array qualitative Bioanalyzer (Agilent, Santa Clara, Calif.) RNA analysis, as previously described (24). The Bioarrays platform v2 contains probes specific to miRNA identified in human, mouse, and rat, as well as additional miRNAs identified through cloning at Ambion, Inc. The Cy5 fluorescence on the arrays was scanned at an excitation wavelength of 635 nm using a GenePix® 4200AL scanner (Molecular Devices, Union City, Calif.). The fluorescent signal associated with the probes and local background was extracted using GenePix® Pro (version 6.0, Molecular Devices). Raw signal data were normalized by first log 2 transformation of signal intensity followed by global Variance Stabilization Normalization (25) of all the arrays within the project. Normalized data were submitted to the GEO archive (accession #GSE11163).

### Quantitative Reverse Transcription-PCR.

TaqMan® miRNA Assays (Applied Biosystems) were used to quantify mature miRNA. cDNA was synthesized by priming with a pool of gene-specific looped primers including the primers of the miRNAs of interest and RNU48, as a universally-expressed endogenous control (Applied Biosystems). 10 μl of total RNA diluted to a final concentration of about 5 ng/μl was used for each reverse transcription (RT) reaction along with other RT components, per manufacturer's specifications. Reactions (about 40 μl) were incubated in an Applied Biosystems GeneAmp® PCR system 9700 for about 30 min at about 16° C., about 30 min at about 42° C., about 5 min at about 85° C., and held at about 4° C. qRT-PCR was performed as previously described (26) with the following exception: all reactions, excluding no-template controls and non-reverse-transcribed controls, were run in triplicate on an ABI 7500 Fast Real Time PCR Detection System. All real-time PCR data were analyzed using the comparative CT method, normalizing against expression of RNU48.

### Statistical Analysis.

Differential expression of miRNAs by microarray was determined with Significance Analysis of Microarray (SAM) software (Stanford University Labs) using 1000 permutations of the data and with delta adjusted to minimize false discovery rate. All hierarchical clustering analyses were carried out using Cluster 3.0 (Stanford University) with Euclidian distance as the distance metric and centroid linkage between clusters. A two-tailed Student's t test was used to compare miRNA expression levels determined by real-time PCR. miRNA expression ratios were calculated following the methods of Gordon et al. (27), and receiver operating curve (ROC) analyses were used to assess the predictive power of the miRNA quotients.

### Unsupervised Hierarchical Clustering Analysis

A microarray platform was used to determine miRNA expression of 662 miRNAs in 16 fresh frozen head and neck squamous cell carcinoma tumors, 5 non-diseased head and neck epithelial tissues, and 2 individual head and neck squamous cell carcinoma cell lines. Unsupervised hierarchical clustering based on all the miRNAs spotted on the chip revealed a marked, very distinct separation of the cell line miRNA profiles compared to those of primary tissues. Additionally, hierarchical clustering based on the limited set of 18 miRNAs determined by SAM analysis to be differentially expressed between tumor and normal showed a clear separation of these two tissue types.

### Results

miRNA expression patterns differentiate head and neck squamous cell carcinoma cell lines from primary tissues.

A microarray platform was used to determine miRNA expression of 662 miRNAs in 16 fresh frozen head and neck squamous cell carcinoma tumors, 5 non-diseased head and neck epithelial tissues, and 2 individual head and neck squamous cell carcinoma cell lines. The normalized data has been deposited in the GEO archive (accession #GSE11163).

Unsupervised hierarchical clustering based on all the miRNAs spotted on the chip revealed a marked, very distinct separation of the cell line miRNA profiles compared to those of primary tissues. SAM identified 67 significantly differentially expressed miRNAs (Q<0.0001) between cell lines and primary tissues, consistently showing lower expression of miRNAs in cell lines compared to tumors (Table 1).

TABLE 1
Differentially expressed miRNA in cell lines vs.
tumors as determined by SAM method (Q < .001).

| miRNA | fold change |
|---|---|
| hsa_miR_143 | −232.76 |
| hsa_miR_145 | −244.22 |
| hsa_miR_199a | −88.23 |
| hsa_miR_199a_AS | −127.06 |
| hsa_miR_146b | −38.49 |
| hsa_miR_223 | −81.81 |
| hsa_miR_214 | −34.36 |
| hsa_miR_126 | −37.12 |
| hsa_miR_34a | −11.68 |
| ambi_miR_3046 | −29.63 |
| ambi_miR_13258 | −9.50 |
| ambi_miR_2660 | −9.35 |
| mmu_miR_10b | −11.09 |
| hsa_miR_10b | −9.04 |
| hsa_miR_142_5p | −9.74 |
| ambi_miR_10411 | −6.13 |
| mmu_miR_451 | −32.32 |
| ambi_miR_7029 | −29.44 |
| hsa_miR_152 | −5.09 |
| mmu_miR_140_AS | −4.53 |
| hsa_miR_451 | −25.01 |
| hsa_miR_199b | −6.69 |
| ambi_miR_13268 | −3.28 |
| hsa_miR_34b | −3.30 |
| rno_miR_140_AS | −3.43 |
| mmu_miR_99a | −10.28 |
| hsa_miR_368 | −6.22 |
| hsa_miR_99a | −9.55 |
| hsa_miR_24 | −2.60 |
| hsa_miR_497 | −4.70 |
| hsa_miR_26b | −2.94 |
| hsa_miR_342 | −3.37 |
| rno_miR_497 | −5.08 |
| hsa_miR_195 | −5.03 |
| ambi_miR_2837 | −4.39 |
| mmu_miR_199b | −4.20 |
| mmu_miR_203 | −22.71 |
| ambi_miR_13205 | −2.90 |
| hsa_miR_22 | −2.41 |
| hsa_miR_203 | −15.66 |
| hsa_miR_150 | −7.22 |
| hsa_miR_146a | −9.06 |
| rno_miR_409_3p | −2.92 |
| hsa_miR_27b | −2.45 |
| ambi_miR_444 | −2.29 |
| ambi_miR_13232 | −2.18 |
| ambi_miR_9651 | −2.70 |
| hsa_miR_27a | −2.09 |
| mmu_miR_379 | −2.49 |
| hsa_miR_125b | −3.76 |
| hsa_miR_455 | −2.02 |
| hsa_miR_452 | −3.79 |
| hsa_miR_29c | −2.98 |
| ambi_miR_9451 | −2.40 |
| hsa_miR_210 | −3.61 |
| ambi_miR_12061 | −2.28 |
| hsa_miR_483 | −1.87 |
| hsa_miR_126_AS | −2.90 |
| ambi_miR_13260 | −2.32 |
| rno_miR_29c_AS | −2.08 |
| mmu_miR_341 | −3.14 |
| hsa_miR_139 | −2.59 |
| rno_miR_382 | −2.24 |
| hsa_miR_189 | −2.07 |
| hsa_miR_326 | −2.02 |
| mmu_miR-487b | −2.45 |

(hsa: Homo sapiens; mmu: Mus musculus; ambi: Ambion; rno: Rattus norvegicus)

18 miRNAs are differentially expressed in head and neck squamous cell carcinoma tumor tissue compared to normal head and neck epithelia.

SAM analysis identified 18 miRNAs to be significantly altered in their expression between non-diseased tissues and primary head and neck squamous cell carcinoma tumors, with 17 being up-modulated and 1 down-modulated in tumors (Q<0.0001) (Table 2). Of the 18 differentially expressed miRNAs, 12 were human miRNAs, four were miRNAs identified on mirVANA™ miRNA Bioarray platform v2 (Ambion Inc. Catalog No. AM 1566V2) and two were mouse orthologues (mmu_miR 503 and mmu_miR 221), both of which have known human counterparts with identical mature sequences. Hierarchical clustering based on this limited set of miRNAs showed clear separation of tumors from normal tissues.

TABLE 2
Differentially expressed miRNAs in tumor vs normal
as determined by SAM method (Q < .001).

| miRNA | fold change |
|---|---|
| ambi_miR_562 | 5.24 |
| hsa_miR_21 | 3.67 |
| ambi_miR_7083 | 5.45 |
| hsa_miR_181d | 2.86 |
| hsa_miR_181b | 2.97 |
| hsa_miR_491 | 5.08 |
| hsa_miR_455 | 2.58 |
| ambi_miR_13258 | 2.56 |
| hsa_miR_18a | 2.78 |
| ambi_miR_11541 | 2.28 |
| hsa_miR_130b | 4.17 |
| mmu_miR_503 | 2.63 |
| hsa_miR_221 | 2.26 |
| hsa_miR_193b | 3.44 |
| hsa_miR_181a | 2.11 |
| mmu_miR_221 | 2.17 |
| hsa_miR_18b | 2.50 |
| hsa_miR_375 | −21.88 |

(hsa: Homo sapiens; mmu: Mus musculus; ambi: Ambion)

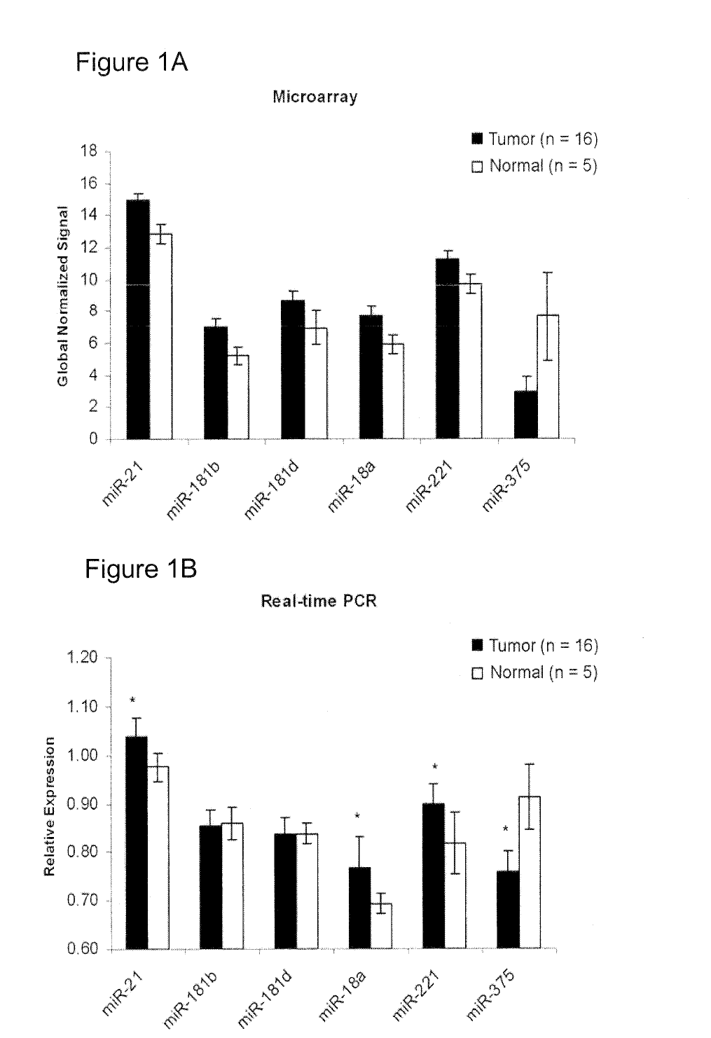

All human miRNAs identified by SAM method showed greater than two-fold difference between normal and tumor tissue (Table 3). Of the human miRNAs, six (miR-21, miR-181d, miR-181b, miR-18a, miR-221, and miR-375) were chosen for confirmation of the microarray results using stem-loop based RT-PCR followed by conventional Taqman real-time PCR using miRNA-specific probes, as validated assays were available for these six miRNA and they constituted one primer pool, allowing for examination using a single reverse transcription step. Confirmation assays utilized only the samples screened on the array. Of the six miRNAs tested, four were reliably confirmed (miR-21, miR-18a, miR-221, and miR-375), showing significant differential expression between tumor and normal (P<0.01, FIGS. 1A and B).

TABLE 3
Annotated human miRNAs differentially expressed
in Tumor vs Normal by SMA (Q < 0.001)

| miRNAs | Fold change |
|---|---|
| miR-21 | 3.67 |
| miR-181d | 2.86 |
| miR-181b | 2.97 |
| miR-491 | 5.08 |
| miR-455 | 2358 |
| miR-18a | 2.78 |
| miR-130b | 4.17 |
| miR-221 | 2.26 |
| miR-193b | 3.44 |
| miR-181a | 2.11 |
| miR-18b | 2.5 |
| miR-375 | −21.88 |

miRNA expression ratio demonstrates high specificity and sensitivity in predicting disease.

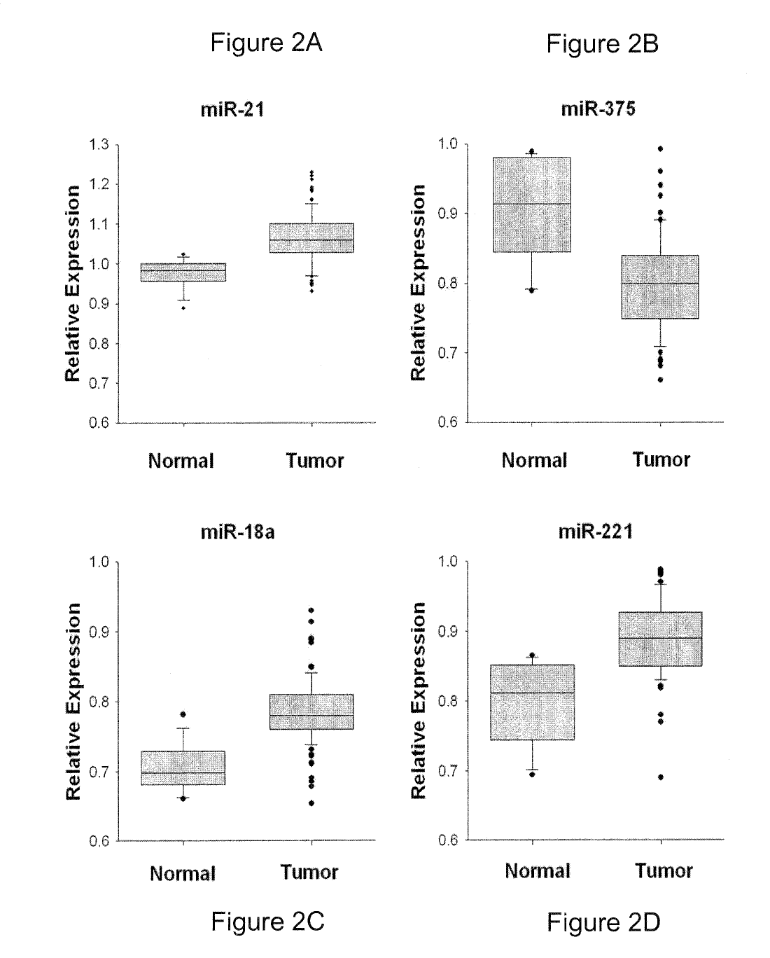

Validation of the four confirmed miRNAs was performed in nine additional normal tissue samples (total n=14), and 83 additional tumor samples (total n=99). Quantification of miRNA expression in this large set of samples showed strong validation of the results seen in the smaller population. miR-21, miR-18a, and miR-221 showed significant upregulation in tumors (P<0.0001; FIGS. 2A, 2C, and 2D, respectively), whereas miR-375 was significantly downregulated in tumors (P<0.0001; FIG. 2B).

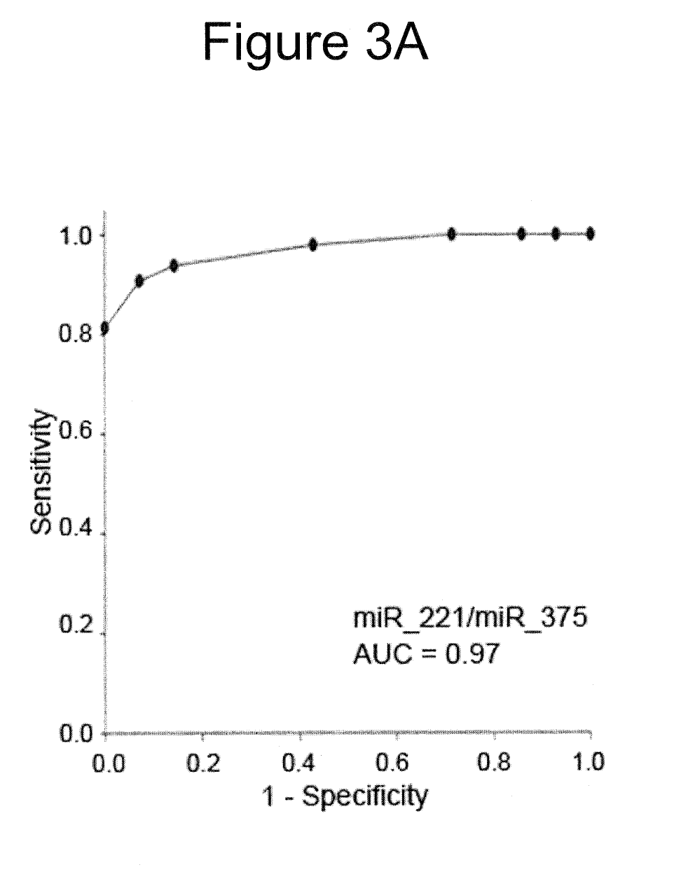

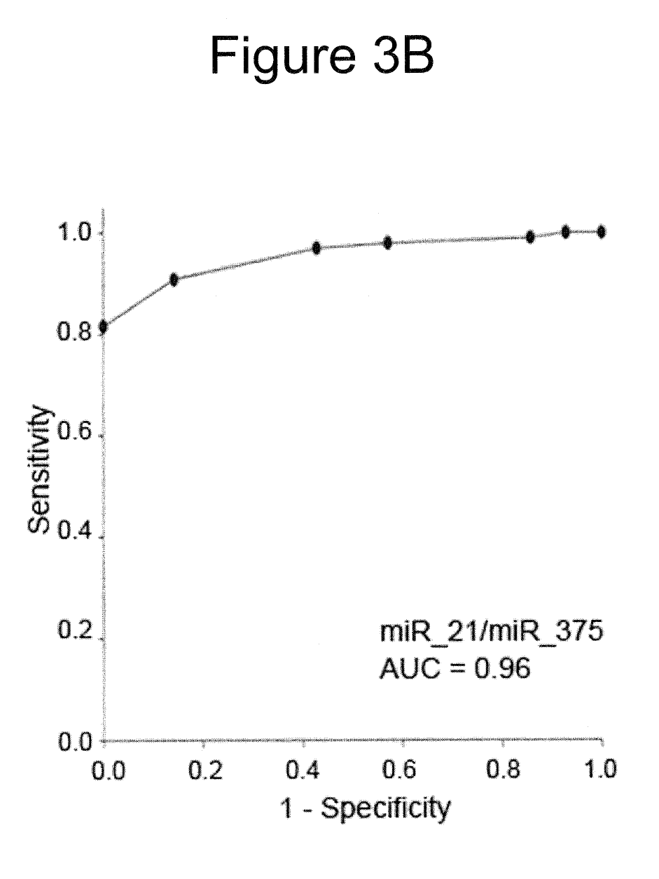

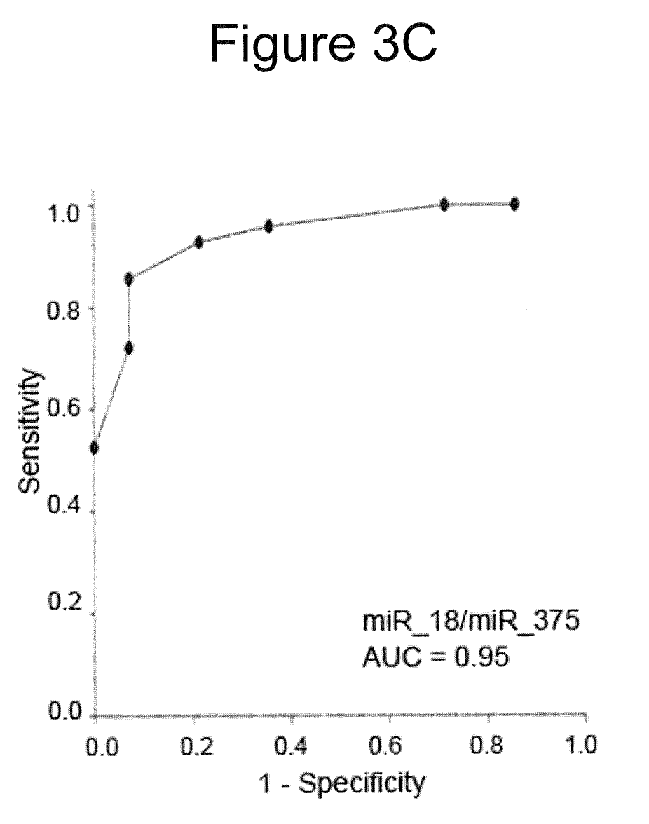

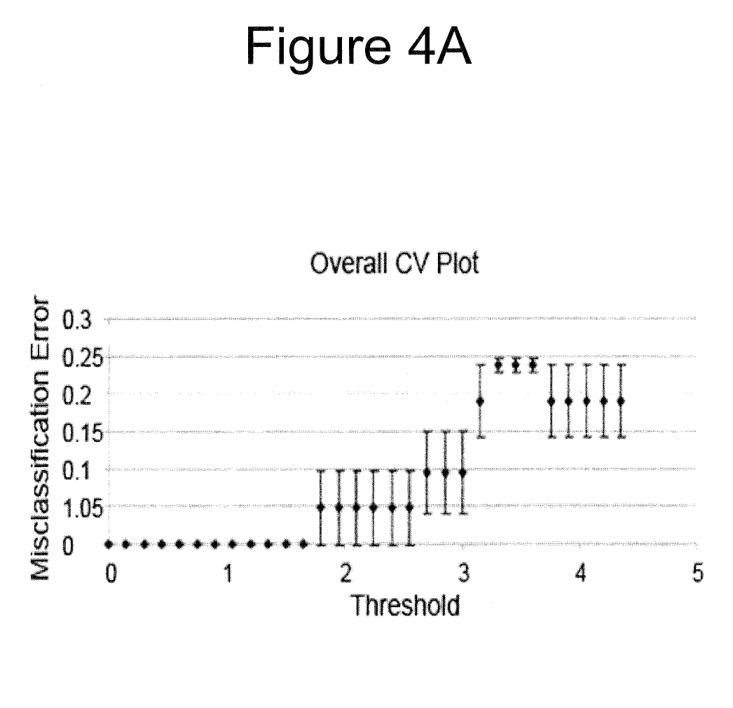

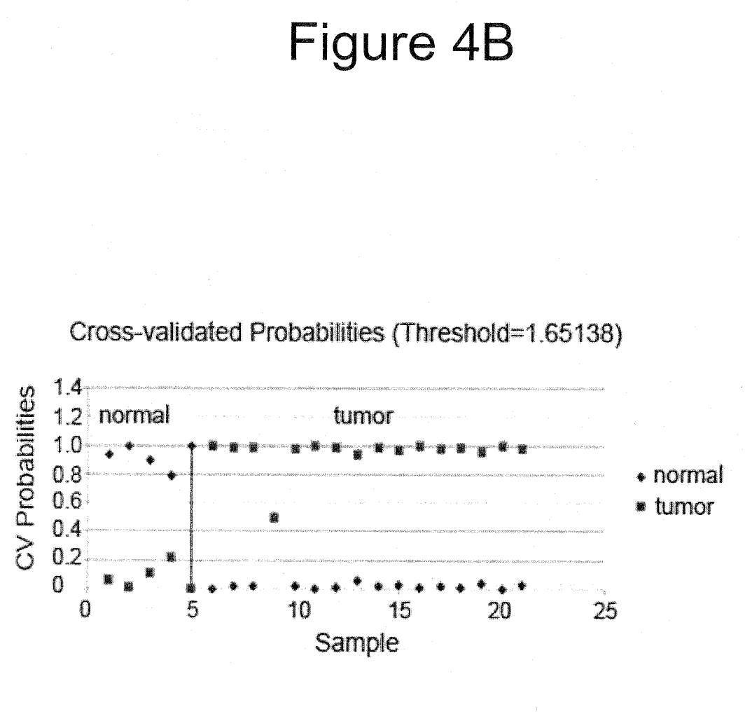

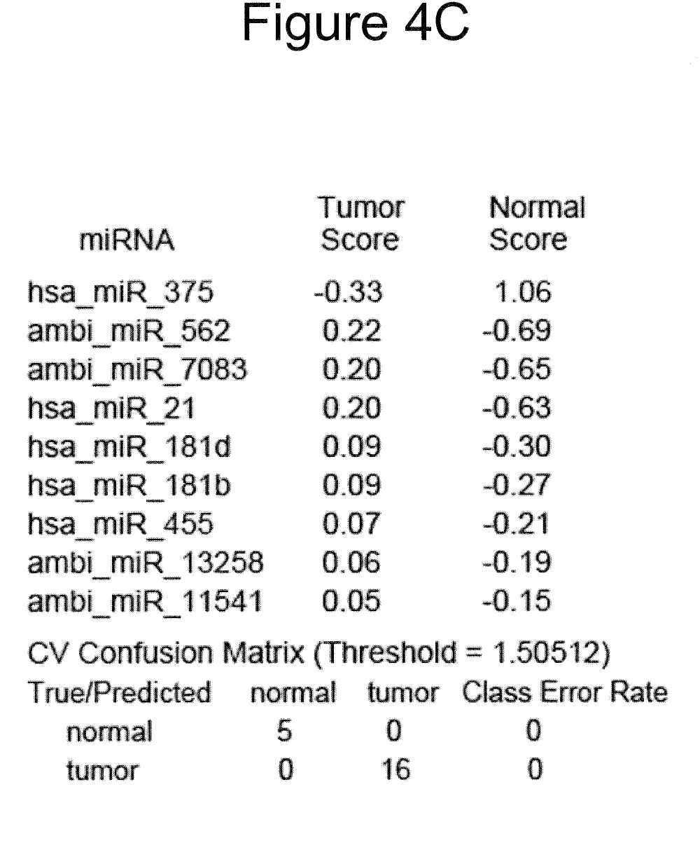

As miR-21, miR-18a, and miR-221 demonstrated consistent upregulation, and miR-375 consistent downregulation, expression ratios were constructed between these miRNAs to determine whether the ratios could improve predictive potential for differentiating head and neck squamous cell carcinoma tumor from non-diseased epithelia. Following the methods of Gordon et al. (27), miRNA expression ratios were calculated by dividing the relative expression value of each of the three miRNAs showing upregulation in tumors by the expression value of the only downregulated miRNA, miR-375. ROC analysis was performed to determine which of these ratios demonstrated the greatest predictive power (FIGS. 3A, 3B and 3C). Table 4 lists the representative sensitivity and specificity of ratios using the cutoff value of 1 for each of the upregulated miRNA to the downregulated miR-375 in differentiating between non-diseased tissue and head and neck squamous cell carcinoma, using the validation series. miR-21:miR-375 ratios above 1.0 exhibited high specificity (0.99) but low sensitivity (0.14) whereas the relationship of miR-18a:miR-375 showed high sensitivity (1.00) but low specificity (0.52) (Table 4). However, the ratio of miR-221:miR-375 exhibited the strongest predictive ability with both high sensitivity and specificity (about 0.92 and about 0.93 respectively; Table 4).

TABLE 4
Examination of sensitivity and specificity of disease prediction
using a miRNA expression ratio greater than 1.0

| Ratio | Sensitivity | Specificity |
|---|---|---|
| miR-21/miR-375 | 0.99 | 0.14 |
| miR-18a/miR-375 | 0.52 | 1 |
| miR-221/miR-375 | 0.92 | 0.93 |

### Discussion

The present study revealed a number of miRNAs to be aberrantly expressed in head and neck squamous cell carcinoma tumors, including an extensively validated subset that may hold utility as clinical biomarkers of disease. Microarray profiling of over 600 miRNAs identified 18 miRNAs that were significantly differentially expressed in tumor tissues compared to analogous non-diseased head and neck epithelia. Of the 12 human miRNAs in this group, 4 miRNA which were validated by qRT-PCR were used for in-depth examination of a larger population of fresh frozen head and neck squamous cell carcinoma tumors and normal head and neck tissue.

The permutation-based software SAM was used to identify differentially expressed genes by pair-wise comparisons between groups of interest. It should be noted that SAM was designed (and may be better suited) for identification of important genes from high density microarrays, as it allows the user to control the number of findings based on a desired false discovery rate (FDR) while avoiding parametric assumptions about the data, inherent in tests such as the Analysis of Variance (ANOVA) (28).

The microarray data revealed that head and neck squamous cell carcinoma cell lines show a distinct pattern of miRNA expression compared to primary tumors. This is consistent with past reports demonstrating a clear segregation of cell lines away from primary tumors upon high-throughput analysis of their miRNA expression (22, 29), suggesting that cell lines have limited utility as a model system for the identification of clinically relevant miRNA biomarkers. There remains, though, significant utility in utilizing in-vitro approaches for defining the biological mechanisms of these miRNAs, with appropriate understanding that the pattern of their expression is markedly different from that observed in the parent primary tissue.

Some of the miRNAs identified as differentially expressed in head and neck squamous cell carcinoma compared to normal tissues have been characterized in past reports, particularly in relation to cancer. Relative levels of miR-21 and other miRNAs have been reported to have prognostic relevance for predicting patient survival in lung cancer (31). Targets of miR-21 may include the tumor suppressor genes tropomyosin 1 (TPM1) and programmed cell death 4 (PDCD4) (32, 33). Additionally, miR-221 and miR-18a, both shown to be upregulated in head and neck squamous cell carcinoma tumors, have previously been implicated in hepatocellular, prostate, and other cancers (34-37).

As described herein, miR-375 was the only downregulated miRNA found when comparing tumors and normal tissues, showing about a 22 fold decrease in tumors. Validation experiments also showed it to be sharply and significantly downregulated in tumors relative to normal tissues. miR-375 has been found to regulate insulin secretion in mice and its downregulation has been implicated in aberrant morphology of pancreatic islet cells in zebrafish (38, 39). Recently, miR-375 downregulation has also been associated with β-catenin mutation in hepatocellular adenoma (40). The downregulation of miR-375 in head and neck squamous cell carcinoma tumors, as shown in this work, is consistent with a possible role for miR-375 in the transcriptional repression of oncogenes, analogous to the regulation of oncogenic KRAS by the let-7 miRNA (41).

The ratio of miR-221:miR-375 demonstrated high discriminatory potential, with a sensitivity of about 92% and specificity of about 93% in distinguishing tumor from normal tissue. These data suggest that the ratio of these miRNAs may hold significant clinical potential to diagnose carcinomas.

The identification of an head and neck squamous cell carcinoma-specific miRNA signature indicates a plausible role for miRNAs in the development or progression of this disease. Finding abnormally expressed miRNAs may prove to be an important step in identifying the specific mechanisms of head and neck squamous cell carcinoma carcinogenesis, as these aberrations may constitute early events in initiation or progression of the disease (42). As such, the utilization of a miRNA expression ratio that distinguishes disease tissue from non-diseased tissue holds potential as a simple, early diagnostic for head and neck squamous cell carcinoma. An examination of these miRNA expression ratios in preneoplastic lesions, early stage tumors, and samples obtained for screening, such as saliva and mouthwash can be done. The expression or the miRNA described herein can also be examined with respect to clinical criteria, such as tumor stage, metastasis and prognosis in a larger series of tumors than what has been examined here. The miRNAs identified for diagnostics described herein may be useful in further understanding the diagnostic potential of the miR-221:miR-375 expression ratio.

### REFERENCES

1. Parkin D M. Global cancer statistics in the year 2000. Lancet Oncol 2001; 2: 533-43.
2. Ragin C C, Modugno F, Gollin S M. The epidemiology and risk factors of head and neck cancer: a focus on human papillomavirus. J Dent Res 2007; 86: 104-14.
Loning T, Ikenberg H, Becker J, Gissmann L, Hoepfer I, zur Hausen H. Analysis of oral papillomas, leukoplakias, and invasive carcinomas for human papillomavirus type related DNA. J Invest Dermatol 1985; 84: 417-20.
4. Furniss C S, McClean M D, Smith J F, et al. Human papillomavirus 16 and head and neck squamous cell carcinoma. Int J Cancer 2007; 120: 2386-92.
5. Hardisson D. Molecular pathogenesis of head and neck squamous cell carcinoma. Eur Arch Otorhinolaryngol 2003; 260: 502-8.
6. Al Moustafa A E, Alaoui-Jamali M A, Batist G, et al. Identification of genes associated with head and neck carcinogenesis by cDNA microarray comparison between matched primary normal epithelial and squamous carcinoma cells. Oncogene 2002; 21: 2634-40.
7. Schlecht N F, Burk R D, Adrien L, et al. Gene expression profiles in HPV-infected head and neck cancer. J Pathol 2007; 213: 283-93.
8. Pyeon D, Newton M A, Lambert P F, et al. Fundamental differences in cell cycle deregulation in human papillomavirus-positive and human papillomavirus-negative head/neck and cervical cancers. Cancer Res 2007; 67: 4605-19.
9. Blenkiron C, Miska E A. miRNAs in cancer: approaches, aetiology, diagnostics and therapy. Hum Mol Genet 2007; 16 Spec No 1: R106-13.
10. Bunz F, Dutriaux A, Lengauer C, et al. Requirement for p53 and p21 to sustain G2 arrest after DNA damage. Science 1998; 282: 1497-501.
11. Cowland J B, Hother C, Gronbaek K. MicroRNAs and cancer. Apmis 2007; 115: 1090-106.
12. Du T, Zamore P D. Beginning to understand microRNA function. Cell Res 2007; 17: 661-3.
13. Miska E A. How microRNAs control cell division, differentiation and death. Curr Opin Genet Dev 2005; 15: 563-8.
14. He H, Jazdzewski K, Li W, et al. The role of microRNA genes in papillary thyroid carcinoma. Proc Natl Acad Sci USA 2005; 102: 19075-80.
15. Murakami Y, Yasuda T, Saigo K, et al. Comprehensive analysis of microRNA expression patterns in hepatocellular carcinoma and non-tumorous tissues. Oncogene 2006; 25: 2537-45.
16. Lu J, Getz G, Miska E A, et al. MicroRNA expression profiles classify human cancers. Nature 2005; 435: 834-8.
17. Chang S S, Jiang W W, Smith I, et al. MicroRNA alterations in head and neck squamous cell carcinoma. Int J Cancer 2008; 123: 2791-7.
18. Tran N, McLean T, Zhang X, et al. MicroRNA expression profiles in head and neck cancer cell lines. Biochem Biophys Res Commun 2007; 358: 12-7.
19. Volinia S, Calin G A, Liu C G, et al. A microRNA expression signature of human solid tumors defines cancer gene targets. Proc Natl Acad Sci USA 2006; 103: 2257-61.
20. Guo Y, Chen Z, Zhang L, et al. Distinctive microRNA profiles relating to patient survival in esophageal squamous cell carcinoma. Cancer Res 2008; 68: 26-33.
21. Iorio M V, Visone R, Di Leva G, et al. MicroRNA signatures in human ovarian cancer. Cancer Res 2007; 67: 8699-707.
22. Blenkiron C, Goldstein L D, Thorne N P, et al. MicroRNA expression profiling of human breast cancer identifies new markers of tumor subtype. Genome Biol 2007; 8: R214.
23. Peters E S, McClean M D, Liu M, Eisen E A, Mueller N, Kelsey K T. The ADH1C polymorphism modifies the risk of squamous cell carcinoma of the head and neck associated with alcohol and tobacco use. Cancer Epidemiol Biomarkers Prev 2005; 14: 476-82.
24. Marsit C J, Eddy K, Kelsey K T. MicroRNA responses to cellular stress. Cancer Res 2006; 66: 10843-8.
25. Huber W, von Heydebreck A, Sultmann H, Poustka A, Vingron M. Variance stabilization applied to microarray data calibration and to the quantification of differential expression. Bioinformatics 2002; 18 Suppl 1: S96-104.
26. Chen C, Ridzon D A, Broomer A J, et al. Real-time quantification of microRNAs by stem-loop RT-PCR. Nucleic Acids Res 2005; 33: e179.
27. Gordon G J, Jensen R V, Hsiao L L, et al. Translation of microarray data into clinically relevant cancer diagnostic tests using gene expression ratios in lung cancer and mesothelioma. Cancer Res 2002; 62: 4963-7.
28. Pan W. A comparative review of statistical methods for discovering differentially expressed genes in replicated microarray experiments. Bioinformatics 2002; 18: 546-54.
29. Szafranska A E, Davison T S, John J, et al. MicroRNA expression alterations are linked to tumorigenesis and non-neoplastic processes in pancreatic ductal adenocarcinoma. Oncogene 2007; 26: 4442-52.
30. Cho W C. OncomiRs: the discovery and progress of microRNAs in cancers. Mol Cancer 2007; 6: 60.
31. Yanaihara N, Caplen N, Bowman E, et al. Unique microRNA molecular profiles in lung cancer diagnosis and prognosis. Cancer Cell 2006; 9: 189-98.
32. Frankel L B, Christoffersen N R, Jacobsen A, Lindow M, Krogh A, Lund A H. Programmed cell death 4 (PDCD4) is an important functional target of the microRNA miR-21 in breast cancer cells. J Biol Chem 2008; 283: 1026-33.
33. Zhu S, Si M L, Wu H, Mo Y Y. MicroRNA-21 targets the tumor suppressor gene tropomyosin 1 (TPM1). J Biol Chem 2007; 282: 14328-36.
34, Fornari F, Gramantieri L, Ferracin M, et al. MiR-221 controls CDKN1C/p57 and CDKN1B/p27 expression in human hepatocellular carcinoma. Oncogene 2008; 27: 5651-61.
35. Galardi S, Mercatelli N, Giorda E, et al. miR-221 and miR-222 expression affects the proliferation potential of human prostate carcinoma cell lines by targeting p27Kip1. J Biol Chem 2007; 282: 23716-24.
36. Nam E J, Yoon H, Kim S W, et al. MicroRNA expression profiles in serous ovarian carcinoma. Clin Cancer Res 2008; 14: 2690-5.
37. Takakura S, Mitsutake N, Nakashima M, et al. Oncogenic role of miR-17-92 cluster in anaplastic thyroid cancer cells. Cancer Sci 2008; 99: 1147-54.
38. Poy M N, Eliasson L, Krutzfeldt J, et al. A pancreatic islet-specific microRNA regulates insulin secretion. Nature 2004; 432: 226-30.
39. Kloosterman W P, Lagendijk A K, Ketting R F, Moulton J D, Plasterk R H. Targeted inhibition of miRNA maturation with morpholinos reveals a role for miR-375 in pancreatic islet development. PLoS Biol 2007; 5: e203.
40. Ladeiro Y, Couchy G, Balabaud C, et al. MicroRNA profiling in hepatocellular tumors is associated with clinical features and oncogene/tumor suppressor gene mutations. Hepatology 2008; 47: 1955-63.
41. Johnson S M, Grosshans H, Shingara J, et al. RAS is regulated by the let-7 microRNA family. Cell 2005; 120: 635-47.
42. Gomes C C, Gomez R S. MicroRNA and oral cancer: future perspectives. Oral Oncol 2008; 44: 910-4.

### Example 2

Quantitative real-time PCR was used to determine the relative expression of four of the miRNAs that were identified in Example 1 as being differentially expressed in head and neck squamous cell carcinomas compared to non-diseased epithelia in a larger, independent case-series of head and neck squamous cell carcinoma tumors (n=169), and examine associations of miRNA expression with exposures and clinical features associated with head and neck squamous cell carcinoma. In multivariate analyses, expression of miR-375 was shown to increase with alcohol consumption (P=0.001), and showed higher expression in tumors of pharyngeal and laryngeal origin compared with oral tumors (P<0.05 and P<0.01, respectively). Additionally, high miR-21 expression was associated with significantly decreased 5-year survival in patients (HR, 1, 68; 95% CI 1.03-2.74). Together, these data suggest that alterations in miRNA expression are related to exposures causal in head and neck cancer and may be useful biomarkers of patient outcome.

### Introduction

Head and neck squamous cell carcinoma, the sixth most common cancer worldwide, arises from various sites in the upper aerodigestive tract (1,2). The intricate anatomy of the primary tumor sites bring about complex patterns of invasion and locoregional spread that have proven difficult to treat (1). These features, along with the frequent occurrence of late-stage diagnosis and second primary tumor formation have contributed to a relatively poor 5-year survival rate that has shown only modest improvement over the last three decades (1,3). The major risk factors for head and neck squamous cell carcinoma include tobacco and alcohol, which can act both independently and synergistically, as well as human papilloma virus (HPV) infection, which is an independent risk factor (4). A complete understanding of how these exposures alter cellular functions and the molecular basis for their risk remains elusive. Understanding the molecular nature of head and neck squamous cell carcinoma carcinogenesis is indispensable for improving early diagnosis, predicting prognosis, and establishing effective therapeutics. While several attempts have been made at defining genetic biomarkers for head and neck squamous cell carcinoma (5-7), epigenetic biomarkers likely contribute considerable clinical and biological utility to treating and understanding this disease as it is clear that epigenetics plays a pivotal role in its development and progression.

The field of epigenetic regulation is being intensely researched and in recent years, important roles for epigenetic alterations in head and neck squamous cell carcinoma carcinogenesis have been revealed (8-10). MicroRNAs (miRNAs), a class of non protein-coding RNAs, are now recognized as critical products of the epigenome, orchestrating events ranging from organogenesis to immunity and they are known to be critical in the development of many diseases, including cancer (11,12). By binding to partially complementary sites in the 3′ untranslated regions of their mRNA targets, miRNAs interfere with mRNA translation or cause mRNA degradation thereby repressing gene expression post-transcriptionally (13).

Dysregulation of miRNAs in cancer has been shown to associate with various tumor characteristics and prognosis in a variety of tumor types (14-17). Though aberrations in miRNA expression in primary head and neck squamous cell carcinoma tumors have recently been defined in several reports (18-21), little is known about how these differences associate with clinical features and disease risk factors. In-vitro studies and animal models have suggested that the expression of miRNAs are altered in response to various toxicant exposures (22-24). Determining such associations in human populations may be vital to better understanding the molecular mechanism through which exposures and the environment contribute to head and neck carcinogenesis. The expression of four miRNAs, previously found to be differentially expressed in head and neck squamous cell carcinoma tumors compared to normal tissues (18), was studied and associations with clinicopathologic features of tumors were examined to determine if these miRNA alterations are useful as prognostic biomarkers. Likewise, associations of expression of these miRNAs with patient carcinogen exposure were investigated in order to better understand if these exposures act via alterations to miRNA.

### Materials and Methods

### Study Population.

A total of 169 fresh-frozen head and neck squamous cell carcinoma tumor samples were obtained, with informed consent after Institutional Review Board approval at participating hospitals, as part of a previously described study of head and neck cancer in the Greater Boston Metropolitan area (25,26). A study pathologist confirmed >75% tumor content in each of the head and neck squamous cell carcinoma samples used in these analyses. HPV-16 DNA status was previously determined (26,27).

### RNA Isolation and Microarray Profiling.

Total RNA was isolated from tumors using the mirVANA RNA Isolation Kit (Ambion, Inc., Austin, Tex.) according to the manufacturer's protocol. RNA was quantified using the Nanodrop ND-1000 spectrophotometer (Nanodrop, Wilmington, Del.), aliquoted, and stored at −80° C. briefly until used in laboratory analysis.

### Quantitative Reverse Transcription-PCR.

TaqMan miRNA Assays (Applied Biosystems, Foster City, Calif.) were used to quantify mature miRNA of miR-21, miR-18a, miR-375, and miR-218. cDNA was synthesized by priming with a pool of gene-specific looped primers including the primers of the miRNAs of interest and RNU48, a universally-expressed endogenous control (Applied Biosystems, Foster City, Calif.). 10 μl of total RNA diluted to a final concentration of 5 ng/μl was used for each reverse transcription (RT) reaction along with other RT components, per manufacturer's specifications. 40 μl reactions were incubated in an Applied Biosystems GeneAmp PCR system 9700 for 30 min at 16° C., 30 min at 42° C., 5 min at 85° C., and held at 4° C. All reactions, excluding no-template controls and non-reverse-transcribed controls, were run in triplicate on an ABI 7500 Fast Real Time PCR Detection System. All real-time PCR data were quantified by calculating fold change using the ΔΔCT method, normalizing miRNA expression of cases to expression data from a pooled set of non-diseased head and neck epithelium samples.

### Statistical Analysis.

All analyses were carried out using SAS 9.1 (SAS Institute, Cary, N.C.). Fold change expression values for all miRNAs were log transformed to create a normal distribution for parametric analysis. Categories of smoking and drinking were created with never smokers/drinkers as referents and low and high categories dichotomized at the median value for each variable. For univariate analyses, student's t-tests or one-way analysis of variance (ANOVA) were used for discrete variables of two or greater than two categories, respectively, except in the case of tumor site, where t-tests were conducted to compare pharyngeal vs oral and laryngeal vs oral tumors. Spearman's rank correlation was used for continuous variables. Linear regression analysis was used for multivariate testing for associations with exposures and clinicopathologic features. Using the Kaplan-Meier method and the log-rank test to determine significance, overall 5-year survival rates were compared across strata of miRNA expression using log-transformed miRNA expression values stratified around the top 25th percentile of miRNA expression. A multivariate Cox proportional hazards regression analysis was used to confirm predictors of case fatality. P-values of <0.05 were considered significant. For best parsimony in multivariate models, all variables were initially included in models but non-significant variables were removed if their removal did not result in greater than 15% change in the effect estimates of other variables (i.e. the variables were not considered confounders).

### Results

Quantification of miRNA Expression.

A subset of 169 patients for which fresh-frozen tumor was available and covariate data were well-annotated was used for this study (Table I). The population consisted of patients with a mean age of 61.5±11.9, 68% of which were males. Greater than half of the participants had smoked more than or equal to 33 pack-years and drank more than or equal to 14 alcoholic drinks per week (51.1% and 51.8%, respectively). The majority of the patients had oral tumors, high stage disease, and were HPV-16 negative (64%, 72%, and 90% respectively).

TABLE I
Patient Demographics and clinicopathological
characteristics (n = 169)

| | n (%) | |
|---|---|---|
| Gender | | |
| Female | 54 | (32.0) |
| Male | 115 | (68.0) |
| Age, mean (SD) | 61.5 | (11.9) |
| Lifetime pack-years smoked^{a} | | |
| 0 (none) | 22 | (15.5) |
| >0 to <36.75 | 59 | (41.5) |
| ≧36.75 | 61 | (43.0) |
| Drinks per week^{b} | | |
| none(0) | 16 | (11.5) |
| >0 to <18 | 61 | (43.9) |
| ≧18 | 62 | (44.6) |
| Tumor Site^{c} | | |
| Oral | 94 | (64.0) |
| Pharyngeal | 31 | (21.1) |
| Laryngeal | 22 | (15.0) |
| Stage^{d} | | |
| Low (I, II) | 46 | (28.0) |
| High (III, IV) | 118 | (72.0) |
| HPV-16 tumor DNA status^{e} | | |
| negative | 90 | (82.6) |
| positive | 19 | (17.4) |

^{a}Data missing in 27 samples
^{b}Data missing in 30 samples
^{c}Data missing in 22 samples
^{d}Data missing in 5 samples
^{e}Data missing in 60 samples

Four miRNAs were selected for analysis in this population based on the results from the study described in Example 1 herein, which identified these miRNAs as differentially expressed between head and neck squamous cell carcinoma tumor and normal head and neck epithelia (18). Quantitative real-time PCR was performed on 169 head and neck squamous cell carcinoma tumors and a fold change expression values of each miRNA was determined by normalizing its expression to a pool of non-diseased samples. The average log-transformed expression values for each of the four miRNAs evaluated are listed in Table Ia. miR-375 expression is associated with tumor site, stage, and alcohol consumption.

In a univariate comparison, miR-375 was expressed at a significantly greater level in laryngeal tumors compared with those of the oral cavity (P=0.004; Table Ia). Tobacco smoking (measured as pack-years smoked, duration (years smoked), or intensity (packs per day)) and HPV status were not associated with miR-375 or any other miRNA's expression. However, univariate analyses showed that the expression of miR-375 increased significantly with alcohol consumption (P=0.009; Table Ia). To control for potential confounders, a multivariate linear regression analysis was employed, which demonstrated that both categories of drinkers, >0-14 and ≦14 drinks per week, were associated with higher miR-375 expression compared to nondrinkers (P=0.052 and P=0.001, respectively; Table II). Further, miR-375 expression was greater in both pharyngeal and laryngeal tumors compared with oral tumors (P=0.047 and P=0.004, respectively; Table II) in a model controlling for age, gender, and tobacco smoking.

TABLE IA
Log-transformed normalized fold-change expression of miRNAs

| microRNA | Mean expression (range) | |
|---|---|---|
| miR-21 | 1.66 | (−2.15, 5.74) |
| miR-375 | −2.3 | (−7.76, 2.82) |
| miR-221 | 1.36 | (−2.36, 6.44) |
| miR-18a | −0.3 | (−3.01, 3.53) |

TABLE II
Univariate analysis of miRNA expression with etiological factors and clinicopathological characteristics^{a}

| | | | log miR21 | | | log miR375 | | | log miR18a | | | log miR221 | | |
|---|---|---|---|---|---|---|---|---|---|---|---|---|---|---|
| | n (%) | | Mean Exp (SD) | | p | Mean Exp (SD) | | p | Mean Exp (SD) | | p | Mean Exp (SD) | | p |
| Gender | | | | | | | | | | | | | | |
| Female | 54 | (32.0) | 1.76 | (1.37) | | −2.49 | (2.04) | | −0.2 | (1.14) | | 1.5 | (1.49) | |
| Male | 115 | (68.0) | 1.64 | (1.47) | 0.61 | −2.20 | (2.14) | 0.41 | −0.33 | (0.94) | 0.41 | 1.31 | (1.40) | 0.42 |
| Age, mean (SD) | 61.5 | (11.9) | 1.68 | (1.44) | 0.21 | −2.3 | (2.10) | 0.63 | −0.29 | (1.01) | 0.29 | 1.37 | (1.42) | 0.12 |
| Lifetime | | | | | | | | | | | | | | |
| pack-years | | | | | | | | | | | | | | |
| smoked | | | | | | | | | | | | | | |
| 0 (none) | 22 | (15.5) | 1.54 | (1.69) | | −2.47 | (2.1) | | −0.20 | (1.3) | | 1.66 | (1.80) | |
| >0 to <36.75 | 59 | (41.5) | 1.63 | (1.40) | | −2.67 | (2.07) | | −0.20 | (0.87) | | 1.35 | (1.29) | |
| ≧36.75 | 61 | (43.0) | 1.64 | (1.28) | 0.96 | −1.98 | (2.27) | 0.21 | −0.44 | (1.00) | 0.37 | 1.28 | (1.39) | 0.55 |
| Drinks per week | | | | | | | | | | | | | | |
| none(0) | 16 | (11.5) | 1.62 | (1.27) | | −3.45 | (1.56) | | −0.21 | (1.00) | | 1.51 | (1.16) | |
| >0 to <18 | 61 | (43.9) | 1.59 | (1.47) | | −2.53 | (2.38) | | −0.26 | (1.09) | | 1.21 | (1.62) | |
| ≧18 | 62 | (44.6) | 1.68 | (1.39) | 0.94 | −1.81 | (1.93) | 0.014 | −0.32 | (0.95) | 0.9 | 1.51 | (1.31) | 0.49 |
| Tumor Site^{b} | | | | | | | | | | | | | | |
| Oral | 94 | (64.0) | 1.62 | (1.49) | | −2.58 | (2.06) | | −0.32 | (1.11) | | 1.37 | (1.54) | |
| Pharyngeal | 31 | (21.1) | 1.46 | (1.36) | 0.11 | −1.78 | (2.52) | 0.12 | −0.36 | (0.82) | 0.81 | 1.14 | (1.25) | 0.41 |
| Laryngeal | 22 | (15.0) | 2.18 | (1.39) | 0.56 | −1.23 | (1.71) | 0.004 | −0.0006 | (0.91) | 0.17 | 1.71 | (1.38) | 0.31 |
| Stage | | | | | | | | | | | | | | |
| Low (I, II) | 46 | (28.0) | 1.74 | (1.36) | | −2.46 | (1.98) | | −0.42 | (1.23) | | 1.02 | (1.3) | |
| High (III, IV) | 118 | (72.0) | 1.60 | (1.47) | 0.58 | −2.36 | (2.11) | 0.77 | −0.38 | (0.98) | 0.78 | 1.15 | (1.24) | 0.31 |
| HPV status | | | | | | | | | | | | | | |
| negative | 90 | (82.6) | 1.73 | (1.33) | | −2.27 | (2.16) | | −0.26 | (1.02) | | 1.53 | (1.29) | |
| positive | 19 | (17.4) | 1.45 | (1.62) | 0.43 | −2.42 | (2.11) | 0.79 | −0.32 | (1.00) | 0.9 | 1.28 | (1.48) | 0.61 |

^{a}Expression values represent log-transformed values obtained by using the ΔΔCT method to normalize data to a pooled referent of non-diseased head and neck epithelium samples.
^{b}T-test results for tumor site compared pharyngeal vs oral and laryngeal vs oral

miR-21 expression is associated with poorer patient survival.

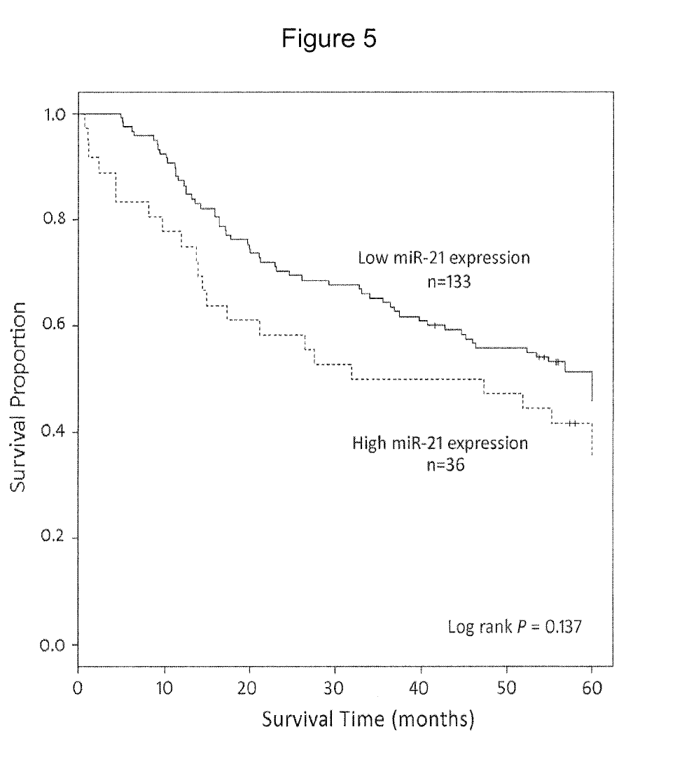

Kaplan-Meier survival analysis demonstrated that patients with miR-21 expression in the highest quartile showed a trend toward worse survival than those with lower miR-21 expression (FIG. 5, P=0.137, log-rank test). However, in a multivariate Cox proportional-hazards model controlling for age, gender and tumor stage, high expression of miR-21 was shown to be associated with significantly decreased 5-year survival of patients (HR=1.68; 95% CI, 1.04-2.77; P=0.034; Table III).

TABLE III
Correlation of miR-375 expression with etiological factors and tumor
site in a multivariate linear regression model (Total n = 133)

| | n (%) | | Reg. Coeff. | p |
|---|---|---|---|---|
| Gender | | | | |
| Female | 42 | (31.6) | reference | |
| Male | 91 | (68.4) | −0.41 | 0.33 |
| Age, mean (SD) | 61.5 | (11.3) | −0.004 | 0.806 |
| Pack-years smoked | | | | |
| none(0) | 22 | (16.5) | reference | |
| <36.75 | 43 | (32.3) | −0.87 | 0.129 |
| ≧36.75 | 68 | (51.1) | −0.5 | 0.375 |
| Drinks per week^{a} | | | | |
| none(0) | 13 | (9.8) | reference | |
| <18 | 50 | (37.6) | 1.3 | 0.039 |
| ≧18 | 70 | (52.6) | 2.26 | 0.002 |
| Tumor Site | | | | |
| Oral | 83 | (62.4) | reference | |
| Pharyngeal | 31 | (23.3) | 0.89 | 0.049 |
| Laryngeal | 19 | (14.3) | 1.7 | 0.005 |

^{a}Trend test P = 0.002

TABLE IV
Cox Proportional Hazards Multivariate Model (n = 150)^{a}

| | | Hazard | 95% CI | | |
|---|---|---|---|---|---|
| | n (%) | Ratio | Lower | Upper | p |
| Gender | | | | | |
| Female | 49 (32.7) | 1.0 | | | |
| | | (reference) | | | |
| Male | 101 (67.3) | 1.24 | 0.76 | 2.01 | 0.388 |
| Age, mean (SD) | | 1.03 | 1.02 | 1.05 | <0.001 |
| log miR-21 | | | | | |
| expression | | | | | |
| <2.58 (n = 117) | 117 | 1.0 | | | |
| | | (reference) | | | |
| ≧2.58 (n = 33) | 33 | 1.68 | 1.04 | 2.77 | 0.034 |
| Tumor stage | | | | | |
| I, II (n = 43) | 43 | 1.0 | | | |
| | | (reference) | | | |
| III, IV (n = 107) | 107 | 1.56 | 0.94 | 2.6 | 0.096 |

^{a}Survival data missing in 15 samples

### Discussion

The potential utility in assessing miRNA expression as a useful biomarker in cancer diagnostics, prognostics and therapeutics, is becoming increasingly apparent, Many studies have reported significant associations between miRNA profiles and important clinical features of tumors as well as patient survival (14,17,28-30), Here, miRNA expression in head and neck squamous cell carcinoma tumors was analyzed and a significant correlation with alcohol consumption, a major head and neck squamous cell carcinoma risk factor, was found, as well as associations with tumor site and overall patient survival.

Four miRNAs were validated as significantly differentially expressed between primary head and neck squamous cell carcinoma tumors and analogous normal tissue (Example 1). These miRNAs may play a role in head and neck squamous cell carcinoma carcinogenesis and that analysis of a large set of tumors would reveal associations between expression of these miRNAs and various clinicopathologic and etiologic variables amongst tumors. Though miR-18a and miR-221 were not found to associate with any covariates in this study, there is good reason to believe that they play a role head and neck squamous cell carcinoma carcinogenesis. miR-18a is a member of the miR-17-92 cluster, found to be overexpressed in gastric, colorectal, and ovarian cancers and is thought to inhibit expression of estrogen receptor-a in hepatocellular carcinoma (31-34). miR-221 expression is also increased in many cancers and its inhibition, along with that of miR-21 has been shown to induce cell-cycle arrest and apoptosis as well as sensitizing cells to chemotherapeutic agents (35,36). Several significant associations with miR-21 and miR-375 were identified in this study. miR-21 is one of the best studied miRNA (37,38). Though miR-375 has mainly been studied in the context of diabetes, as it influences beta-cell mass and insulin levels (39,40), it's expression has been shown to be decreased in a number of malignancies including pancreatic adenocarcinomas and esophageal squamous cell and adenocarcinomas (41,42). Additionally, the recent identification of a target for miR-375, phosphoinosidtide-dependent protein kinase-1 (PDPK1), suggests a feasible role for miR-375 as a tumor suppressor since PDPK1 is crucial for the activation of anti-apoptotic AKT (43).

Alcohol consumption has been associated with altered miRNA expression in hepatocellular tumors and alcohol treatment has been shown to affect miRNA levels in rat neurons and fetal mouse brains (44-46). miR-375 expression was shown to increase with increasing alcohol consumption independent from tobacco smoking. While it is known that alcohol is a carcinogen and an independent risk factor for head and neck squamous cell carcinoma, the mechanism for this association is poorly understood. One possibility is that alcohol acts as an irritant and the resultant inflammation contributes to carcinogenesis (47). The oxidation of ethanol in the saliva by mucosal and microbial alcohol dehydrogenases results in the production of acetaldehyde, which is a known carcinogen in animals and possible carcinogen in humans (48,49). Additionally, it is well established that alcohol interferes with the absorption of folate, a methyl-donor critical for maintenance of normal DNA methylation patterns (50). Although miR-375 is down-regulated in tumors compared to normal tissues, alcohol consumption could contribute to its altered expression in the tumor microenvironment. The regulation of miRNAs is complex and perturbations of the normal homeostatic mechanisms responsible for overall epigenetic stability could play a crucial role in potentially carcinogenic gene expression.

Higher expression of miR-375 was also found in pharyngeal and laryngeal tumors compared with tumors of the oral cavity. This observation is consistent with findings indicating that miRNA profiles are tumor and cell-type specific and can even precisely differentiate tumor subtypes (51,52). Moreover, the proclivity for differential expression of miR-375 in tissues might reflect etiology. The significant association observed between drinking and miR-375 expression coupled with its tendency for higher expression in pharyngeal and laryngeal tumors may suggest that the dysregulation of miRNA by exposures occurs preferentially in certain tissues.

This study has identified a significant association between high miR-21 expression in tumors and poor patient survival, the same relationship that has been demonstrated in cancers of the breast and colon as well as in non-small cell lung cancer (15,16,53). Several targets of miR-21 have been experimentally validated, many of which are tumor-suppressor genes (54-56). One of these targets, programmed cell death 4 (PDCD4) is known to be down-regulated in head and neck squamous cell carcinoma and in two recent studies of miRNA profiles in head and neck squamous cell carcinoma, its expression was shown to be inversely related to miR-21 in tumors (20,21). Another important target which shows reduced expression in head and neck squamous cell carcinoma is PTEN, a gene whose product inhibits growth and cell survival through antagonism of the AKT/PI3K pathway (57). Thus, it is likely that miR-21 functions through several targets to contribute to head and neck squamous cell carcinoma malignancy, thereby modifying risk associated with the disease.

The results in this Example suggest that miRNAs, such as miR-375, may modulate the carcinogenic response associated with exposure to risk factors for the disease. Further, this modulation may be differentially regulated in tumors depending on the tissue, as the expression of miR-375 was shown to differ amongst tumor sites. More in-depth study of miR-375 may prove invaluable for understanding of how exposures modify risk or progression of head and neck squamous cell carcinoma. Additionally, high miR-21 expression correlated with poor prognosis in head and neck squamous cell carcinoma patients. As miR-21 seems to be a significant indicator of prognosis for this and other cancers, it should be considered as a potential therapeutic target for these diseases. The results in this Example also suggest there may be significant prognostic utility in examining these specific miRNA expression signatures.

### REFERENCES

1. Chin, D., Boyle, G. M., Porceddu, S., Theile, D. R., Parsons, P. G. and Coman, W. B. (2006) Head and neck cancer: past, present and future. Expert Rev Anticancer Ther, 6, 1111-8.
2. Parkin, D. M., Bray, F., Ferlay, J. and Pisani, P. (2005) Global cancer statistics, 2002. CA Cancer J Clin, 55, 74-108.
3. Jemal, A., Siegel, R., Ward, E., Hao, Y., Xu, J., Murray, T. and Thun, M. J. (2008) Cancer statistics, 2008, CA Cancer J Clin, 58, 71-96.
4. Ragin, C. C., Modugno, F. and Gollin, S. M. (2007) The epidemiology and risk factors of head and neck cancer: a focus on human papillomavirus. J Dent Res, 86, 104-14.
5. Lothaire, P., de Azambuja, E., Dequanter, D., Lalami, Y., Sotiriou, C., Andry, G., Castro, G., Jr. and Awada, A. (2006) Molecular markers of head and neck squamous cell carcinoma: promising signs in need of prospective evaluation. Head Neck, 28, 256-69.
6. Thomas, G. R., Nadiminti, H. and Regalado, J. (2005) Molecular predictors of clinical outcome in patients with head and neck squamous cell carcinoma, Int J Exp Pathol, 86, 347-63.
7. Chen, C., Mendez, E., Houck, J., Fan, W., Lohavanichbutr, P., Doody, D., Yueh, B., Futran, N. D., Upton, M., Farwell, D. G., Schwartz, S. M. and Zhao, L. P. (2008) Gene expression profiling identifies genes predictive of oral squamous cell carcinoma. Cancer Epidemiol Biomarkers Prev, 17, 2152-62.
8. Fan, C. Y. (2004) Epigenetic alterations in head and neck cancer: prevalence, clinical significance, and implications. Curr Oncol Rep, 6, 152-61.
9. Marsit, C. J., Christensen, B. C., Houseman, E. A., Karagas, Wrensch, M. R., Yeh, R. F., Nelson, H. H., Wiemels, L E, Zheng, S., Posner, M. R., McClean, M. D., Wiencke, J. K. and Kelsey, K. T. (2009) Epigenetic profiling reveals etiologically distinct patterns of DNA methylation in head and neck squamous cell carcinoma. Carcinogenesis, 30, 416-22.
10. Worsham, M. J., Chen, K. M., Meduri, V., Nygren, A. O., Errami, A., Schouten, J. P. and Benninger, M. S. (2006) Epigenetic events of disease progression in head and neck squamous cell carcinoma. Arch Otolaryngol Head Neck Surg, 132, 668-77.
11. Schickel, R., Boyerinas, B., Park, S. M. and Peter, M. E. (2008) MicroRNAs: key players in the immune system, differentiation, tumorigenesis and cell death. Oncogene, 27, 5959-74.
12. Ambros, V. (2004) The functions of animal microRNAs. Nature, 431, 350-5.
13. Cullen, B. R. (2004) Transcription and processing of human microRNA precursors. Mol Cell, 16, 861-5.
14. Calin, G. A., Ferracin, M., Cimmino, A., Di Leva, G., Shimizu, M., Wojcik, S. E., Iorio, M. V., Visone, R., Sever, N J., Fabbri, M., Iuliano, R., Palumbo, T., Pichiorri, F., Roldo, C., Garzon, R., Sevignani, C., Rassenti, L., Alder, H., Volinia, S., Liu, C. G., Kipps, T. J., Negrini, M. and Croce, C. M. (2005) A MicroRNA signature associated with prognosis and progression in chronic lymphocytic leukemia. N Engl J Med, 353, 1793-801.
15. Yan, L. X., Huang, X. F., Shao, Q., Huang, M. Y., Deng, L., Wu, Q. L., Zeng, Y. X. and Shao, J. Y. (2008) MicroRNA miR-21 overexpression in human breast cancer is associated with advanced clinical stage, lymph node metastasis and patient poor prognosis. RNA, 14, 2348-60.
16. Schetter, A. J., Leung, S. Y., Sohn, J. J., Zanetti, K. A., Bowman, E. D., Yanaihara, N., Yuen, S. T., Chan, T. L., Kwong, D. L., Au, G. K., Liu, C. G., Calin, G. A., Croce, C. M. and Harris, C. C. (2008) MicroRNA expression profiles associated with prognosis and therapeutic outcome in colon adenocarcinoma. JAMA, 299, 425-36.
17. Chen, Y. and Stallings, R. L. (2007) Differential patterns of microRNA expression in neuroblastoma are correlated with prognosis, differentiation, and apoptosis. Cancer Res, 67, 976-83.
18. Avissar, M., Christensen, B. C., Kelsey, K. T. and Marsit, C. J. (2009) MicroRNA Expression Ratio Is Predictive of Head and Neck Squamous Cell Carcinoma. Clin Cancer Res, 15, 2850-5.
19. Chang, S. S., Jiang, W. W., Smith, I., Poeta, L. M., Begum, S., Glazer, C., Shan, S., Westra, W., Sidransky, D. and Califano, J. A. (2008) MicroRNA alterations in head and neck squamous cell carcinoma. Int J Cancer, 123, 2791-2797.
20. Childs, G., Fazzari, M., Kung, G., Kawachi, N., Brandwein-Gensler, M., McLemore, M., Chen, Q., Burk, R. D., Smith, R. V., Prystowsky, M. B., Belbin, T. J. and Schlecht, N. F. (2009) Low-level expression of microRNAs let-7d and miR-205 are prognostic markers of head and neck squamous cell carcinoma. Am J Pathol, 174, 736-45.
21. Ramdas, L., Giri, U., Ashorn, C. L., Coombes, K. R., El-Naggar, A., Ang, K. K. and Story, M. D. (2009) miRNA expression profiles in head and neck squamous cell carcinoma and adjacent normal tissue. Head Neck, 31, 642-54.
22. Chaudhry, M. A. (2009) Real-time PCR analysis of micro-RNA expression in ionizing radiation-treated cells. Cancer Biother Radiopharm, 24, 49-56.
23. Marsit, C. J., McClean, M. D., Furniss, C. S. and Kelsey, K. T. (2006) Epigenetic inactivation of the SFRP genes is associated with drinking, smoking and HPV in head and neck squamous cell carcinoma. Int J Cancer, 119, 1761-6.
24, Zhang, B. and Pan, X. (2009) RDX induces aberrant expression of microRNAs in mouse brain and liver. Environ Health Perspect, 117, 231-40.
25, Peters, E. S., McClean, M. D., Liu, M., Eisen, B. A., Mueller, N. and Kelsey, K. T. (2005) The ADH1C polymorphism modifies the risk of squamous cell carcinoma of the head and neck associated with alcohol and tobacco use. Cancer Epidemiol Biomarkers Prev, 14, 476-82.
26. Furniss, C. S., Marsit, C. J., Houseman, E. A., Eddy, K. and Kelsey, K. T. (2008) Line region hypomethylation is associated with lifestyle and differs by human papillomavirus status in head and neck squamous cell carcinomas. Cancer Epidemiol Biomarkers Prev, 17, 966-71.
27. Furniss, C. S., McClean, M. D., Smith, J. F., Bryan, J., Nelson, H. H., Peters, E. S., Posner, M. R., Clark, J. R., Eisen, E. A. and Kelsey, K. T. (2007) Human papillomavirus 16 and head and neck squamous cell carcinoma. Int J Cancer, 120, 2386-92.
28. Slaby, O., Svoboda, M., Fabian, P., Smerdova, T., Knoflickova, D., Bednarikova, M., Nenutil, R. and Vyzula, R. (2007) Altered expression of miR-21, miR-31, miR-143 and miR-145 is related to clinicopathologic features of colorectal cancer. Oncology, 72, 397-402.
29. Yanaihara, N., Caplen, N., Bowman, E., Seike, M., Kumamoto, K., Yi, M., Stephens, R. M., Okamoto, A., Yokota, J., Tanaka, T., Calin, G. A., Liu, C. G., Croce, C. M. and Harris, C. C. (2006) Unique microRNA molecular profiles in lung cancer diagnosis and prognosis. Cancer Cell, 9, 189-98.
30. Yu, S. L., Chen, H. Y., Chang, G. C., Chen, C. Y., Chen, H. W., Singh, S., Cheng, C. L., Yu, C. J., Lee, Y. C., Chen, H. S., Su, T. J., Chiang, C. C., Li, H. N., Hong, Q. S., Su, H. Y., Chen, C. C., Chen, W. J., Liu, C. C., Chan, W. K., Li, K. C., Chen, J. J. and Yang, P. C. (2008) MicroRNA signature predicts survival and relapse in lung cancer. Cancer Cell, 13, 48-57.
31. Liu, W. H., Yeh, S. H., Lu, C. C., Yu, S. L., Chen, H. Y., Lin, C. Y., Chen, D. S. and Chen, P. J. (2009) MicroRNA-18a prevents estrogen receptor-alpha expression, promoting proliferation of hepatocellular carcinoma cells. Gastroenterology, 136, 683-93.
32. Guo, J., Miao, Y., Xiao, B., Huan, R., Jiang, Z., Meng, D. and Wang, Y. (2009) Differential expression of microRNA species in human gastric cancer versus non-tumorous tissues. J Gastroenterol Hepatol, 24, 652-7.
33. Motoyama, K., Inoue, H., Takatsuno, Y., Tanaka, F., Mimori, K., Uetake, H., Sugihara, K. and Mori, M. (2009) Over- and under-expressed microRNAs in human colorectal cancer. Int J Oncol, 34, 1069-75.
34. Nam, E. J., Yoon, H., Kim, S. W., Kim, H., Kim, Y. T., Kim, J. H., Kim, J. W. and Kim, S. (2008) MicroRNA expression profiles in serous ovarian carcinoma. Clin Cancer Res, 14, 2690-5.
35. Park, J. K., Lee, E. J., Esau, C. and Schmittgen, T. D. (2009) Antisense Inhibition of microRNA-21 or -221 Arrests Cell Cycle, Induces Apoptosis, and Sensitizes the Effects of Gemcitabine in Pancreatic Adenocarcinoma. Pancreas.
36. Zhang, C., Kang, C., You, Y., Pu, P., Yang, W., Zhao, P., Wang, G., Zhang, A., Jia, Z., Han, L. and Jiang, H. (2009) Co-suppression of miR-221/222 cluster suppresses human glioma cell growth by targeting p27kip1 in vitro and in vivo. Int J Oncol, 34, 1653-60.
37. Krichevsky, A. M. and Gabriely, G. (2009) miR-21: a small multi-faceted RNA. Cell Mol Med, 13, 39-53.
38. Selcuklu, S. D., Donoghue, M. T. and Spillane, C. (2009) miR-21 as a key regulator of oncogenic processes. Biochem Soc Trans, 37, 918-25.
39. Poy, M. N., Eliasson, L., Krutzfeldt, J., Kuwajima, S., Ma, X., Macdonald, P. E., Pfeffer, S., Tuschl, T., Rajewsky, N., Rorsman, P. and Stoffel, M. (2004) A pancreatic islet-specific microRNA regulates insulin secretion. Nature, 432, 226-30.
40. Poy, M. N., Hausser, J., Trajkovski, M., Braun, M., Collins, S., Rorsman, P., Zavolan, M. and Stoffel, M. (2009) miR-375 maintains normal pancreatic alpha- and beta-cell mass. Proc Natl Acad Sci USA, 106, 5813-8.
41, Mathe, E. A., Nguyen, G. H., Bowman, E. D., Zhao, Y., Budhu, A., Schetter, A. J., Braun, R., Reimers, M., Kumamoto, K., Hughes, D., Altorki, N. K., Casson, A. G., Liu, C. G., Wang, X. W., Yanaihara, N., Hagiwara, N., Dannenberg, A. J., Miyashita, M., Croce, C. M. and Harris, C. C. (2009) MicroRNA expression in squamous cell carcinoma and adenocarcinoma of the esophagus: associations with survival. Clin Cancer Res, 15, 6192-200.
42, Bloomston, M., Frankel, W. L., Petrocca, F., Volinia, S., Alder, H., Hagan, J. P., Liu, C. G., Bhatt, D., Taccioli, C. and Croce, C. M. (2007) MicroRNA expression patterns to differentiate pancreatic adenocarcinoma from normal pancreas and chronic pancreatitis. JAMA, 297, 1901-8.
43. El Ouaamari, A., Baroukh, N., Martens, G. A., Lebrun, P., Pipeleers, D. and van Obberghen, E. (2008) miR-375 targets 3′-phosphoinositide-dependent protein kinase-1 and regulates glucose-induced biological responses in pancreatic beta-cells. Diabetes, 57, 2708-17.
44. Pietrzykowski, A. Z., Friesen, R. M., Martin, G. E., Puig, S. I., Nowak, C. L., Wynne, P. M., Siegelmann, H. T. and Treistman, S. N. (2008) Posttranscriptional regulation of BK channel splice variant stability by miR-9 underlies neuroadaptation to alcohol. Neuron, 59, 274-87.
45. Wang, L. L., Zhang, Z., Li, Q., Yang, R., Pei, X., Xu, Y., Wang, J., Zhou, S. F. and Li, Y. (2009) Ethanol exposure induces differential microRNA and target gene expression and teratogenic effects which can be suppressed by folic acid supplementation. Hum Reprod, 24, 562-79.
46. Ladeiro, Y., Couchy, G., Balabaud, C., Bioulac-Sage, P., Pelletier, L., Rebouissou, S, and Zucman-Rossi, J. (2008) MicroRNA profiling in hepatocellular tumors is associated with clinical features and oncogene/tumor suppressor gene mutations. Hepatology, 47, 1955-63.
47. Seitz, H. K. and Stickel, F. (2007) Molecular mechanisms of alcohol-mediated carcinogenesis. Nat Rev Cancer, 7, 599-612.
48. Homann, N., Tillonen, J., Meurman, J. H., Rintamaki, H., Lindqvist, C., Rautio, M., Jousimies-Somer, H. and Salaspuro, M. (2000) Increased salivary acetaldehyde levels in heavy drinkers and smokers: a microbiological approach to oral cavity cancer. Carcinogenesis, 21, 663-8.
49. Muto, M., Hitomi, Y., Ohtsu, A., Shimada, H., Kashiwase, Y., Sasaki, H., Yoshida, S, and Esumi, H. (2000) Acetaldehyde production by non-pathogenic Neisseria in human oral microflora: implications for carcinogenesis in upper aerodigestive tract. Int J Cancer, 88, 342-50.
50. Hamid, A., Wani, N. A. and Kaur, J. (2009) New perspectives on folate transport in relation to alcoholism-induced folate malabsorption—association with epigenome stability and cancer development. FEBS J, 276, 2175-91.
51. Iorio, M. V., Visone, R., Di Leva, G., Donati, V., Petrocca, F., Casalini, P., Taccioli, C., Volinia, S., Liu, C. G., Alder, H., Calin, G. A., Menard, S, and Croce, C. M. (2007) MicroRNA signatures in human ovarian cancer. Cancer Res, 67, 8699-707.
52. Lu, J., Getz, G., Miska, E. A., Alvarez-Saavedra, E., Lamb, J., Peck, D., Sweet-Cordero, A., Ebert, B. L., Mak, R. H., Ferrando, A. A., Downing, J. R., Jacks, T., Horvitz, H. R. and Golub, T. R. (2005) MicroRNA expression profiles classify human cancers. Nature, 435, 834-8.
53. Markou, A., Tsaroucha, E. G., Kaklamanis, L., Fotinou, M., Georgoulias, V. and Lianidou, E. S. (2008) Prognostic value of mature microRNA-21 and microRNA-205 overexpression in non-small cell lung cancer by quantitative real-time RT-PCR. Clin Chem, 54, 1696-704.
54. Frankel, L. B., Christoffersen, N. R., Jacobsen, A., Lindow, M., Krogh, A. and Lund, A. H. (2008) Programmed cell death 4 (PDCD4) is an important functional target of the microRNA miR-21 in breast cancer cells. J Biol Chem, 283, 1026-33.
55. Li, T., Li, D., Sha, J., Sun, P. and Huang, Y. (2009) MicroRNA-21 directly targets MARCKS and promotes apoptosis resistance and invasion in prostate cancer cells. Biochem Biophys Res Commun.
56. Meng, F., Henson, R., Wehbe-Janek, H., Ghoshal, K., Jacob, S. T. and Patel, T. (2007) MicroRNA-21 regulates expression of the PTEN tumor suppressor gene in human hepatocellular cancer. Gastroenterology, 133, 647-58.
57. Pedrero, J. M., Carracedo, D. G., Pinto, C. M., Zapatero, A. H., Rodrigo, J. P., Nieto, C. S. and Gonzalez, M. V. (2005) Frequent genetic and biochemical alterations of the PI 3-K/AKT/PTEN pathway in head and neck squamous cell carcinoma. Int J Cancer, 114, 242-8.

### Example 3

MicroRNAs (miRNA) are small non-coding RNA molecules, which influence biological functions through their interactions with multiple targets. Overexpression studies have been indispensable for the validation of miRNA targets and determination of cellular functions. The study described in Example 1 herein shows that miR-375 expression is down-regulated in head and neck squamous cell carcinoma. miR-375 may function as a tumor suppressor in head and neck squamous cell carcinoma carcinogenesis. The effects on proliferation, migration and sensitivity to cisplatin (CDDP), were assessed in cells transfected with mature miR-375 mimic molecules and those transfected with a plasmid vector carrying the premature miR-375 sequence under the control of a CMV promoter. miR-375 mimic transfection resulted in >2,000 fold upregulation in miR-375 levels, faster proliferation and migration and increased resistance to CDDP in miR-375 transfected cells. On the other hand, the miR-375 expression vector-based transfection only increased miR-375 expression by ˜4 fold and caused slower proliferation and increased sensitivity to CDDP. These results have shown that different methods of miRNA overexpression vary considerably in their resultant biological effects perhaps due to disparities in the extent to which they analogize endogenous miRNAs. As such, the caveats of their use in such studies must be carefully considered.

### Introduction

MicroRNAs (miRNA) are now known to be important post-transcriptional regulators of gene expression, orchestrating diverse molecular functions through their mRNA targets (1). Primary miRNA transcripts (pri-miRNA) of 100s to 1000s of base pairs in length are transcribed from intergenic regions or intronic sequences into large stem-loop structures which are sequentially processed first generating a ˜70 bp hairpin, which is exported out of the nucleus, and then a mature ˜22 bp duplex (2). The active strand of the duplex is incorporated into a ribonucleoprotein effector complex known as RNA-induced silencing complex (RISC) in the cytoplasm. The miRNA/RISC can then target a range of partially or fully complementary mRNA transcripts, resulting either in their translational repression or degradation, respectively (2).

An involvement of miRNAs has been identified for almost all major cancers (3). Many of the miRNAs significantly altered in cancer tend to target genes that regulate cell proliferation, differentiation and death, disruptions in which are classically associated with malignancy. Previous work showed miR-375, a miRNA primarily associated with pancreatic islet cells (4), to be down-regulated in head and neck squamous cell carcinoma compared to non-diseased tissue (5). miR-375 has been shown to target 3′-phosphoinositide-dependent protein kinase-1 (PDPK-1) in pancreatic beta cells (6). As PDPK-1 is a major activator of anti-apoptotic AKT (7), it is possible that miR-375 down-regulation in head and neck squamous cell carcinoma potentiates a pro-survival carcinogenic phenotype.

The growing interest in miRNAs' involvement in disease has spurred the development of myriad systems designed to study their expression levels, identify targets, and define their pleiotropic roles in vitro as well as in vivo (8). Given their diminutive size, and the fact that they do not code for proteins, the detection of miRNAs has required customized technologies including the development of specialized miRNA arrays and adaptation of traditional real-time PCR techniques for their quantification. The examination of miRNA functions in cultured cell lines carries several caveats as their expression is generally lower in cell lines compared to analogous primary tissues (9,10).

Here several experiments were performed to test changes in cell proliferation, migration and sensitivity to cisplatin, a chemotherapeutic agent commonly used in the treatment of head and neck squamous cell carcinoma (HNSCC). Two different systems were used for overexpression of miR-375 in the invasive head and neck squamous cell carcinoma cell line FaDu. The first system involves transient transfection with a synthetic double-stranded RNA molecule designed to mimic the mature miRNA. Functioning much like small interfering RNAs (siRNAs), these miRNA precursors are chemically modified to ensure that the appropriate strand becomes incorporated into the RISC. The second method utilizes a vector-based system to allow expression of an engineered miRNA sequence from a Pol II promoter. This expression vector contains the human cytomegalovirus (CMV) immediate early promoter, allowing for constitutive miRNA expression in mammalian cells. Both systems have advantages caveats to their use and this study aims to investigate which system is better suited for determination of miRNA function and which most closely reproduces miRNAs generated endogenously.

### Materials and Methods

### Cell Lines

FaDu cells were obtained from ATCC(HTB-43) and cultured in Eagles's Minimum Essential Medium (Invitrogen) supplemented with 10% fetal bovine serum and 1% penicillin-streptomycin.

Reverse Transfection of miRNA Mimics

Pre-miR™ miRNA Precursor Molecules (Ambion) were transfected into cells according to manufacturer's instructions. Briefly, 0.24 μl or 2.4 μl of precursor and 0.45 or 4.5 μl of NeoFX Transfection Agent (Ambion) were diluted in Opti-MEM Reduced Serum Medium, combined and applied to cells while passaging. Normal growth medium was replaced after about 24 hours and cells were used for experiments after another 24 hours.

Creation and Transfection of miR-375 Expression Constructs

Single stranded oligonucleotides containing the premature miR-375 sequence were designed as follows

sense,
(SEQ ID NO: 40)
5'-TGCTGCCCCGCGACGAGCCCC-
TCGCACAAACCGGACCTGAGCGTTTTGTTCGTTCGGCTCGCGTGAGGC-
3';
and
antisense,
(SEQ ID NO: 41)
5'-CCTGGCCTCACGCGAGCCGAACGAACAAAACGCTCAGGTCCGGTTTG
TGCGAGGGGCTCGTCGCGGGGC-3'.

The oligos were annealed and the double-stranded oligo was ligated into the Block-iT™ Pol II miR RNAi Expression Vector according to the manufacturer's instructions (Invitrogen). Both miR-375 and negative control plasmids were transformed into TOP10 Competent E. coli using One Shot TOP10 Transformation Protocol (Invitrogen). Transformants were analyzed by sequencing and successful clones expanded, and their plasmids purified by maxi-prep (Qiagen). FaDu cells were seeded to about 90% confluency in 6-well plates the day before transfection. A vector containing either miR-375 or the negative control miRNA plasmid was transfected into cells by applying 10 μl Lipofectamine-2000 (Invitrogen) and 4 μg plasmid DNA, each diluted in Opti-MEM Reduced Serum Medium (Invitrogen) and combined. Medium was changed to normal growth medium after 4 hours incubation. Vectors contained the coding sequence of EmGFP (Emerald Green Fluorescent Protein) such that the pre-miRNA insertion site is in the 3′ untranslated (3′UTR) region of the fluorescent protein mRNA, allowing for determination of transfection efficiency. Cells were used for experiments about 24 hours after transfection.

### RNA Isolation and Quantitative Real-Time PCR

Total RNA was extracted using mirVana miRNA Isolation Kit (Ambion Inc.) according to manufacturer's protocol. Reverse Transcription PCR using a GeneAmp PCR System 9700 (Applied Biosystems) was performed using 5× primers for hsa-miR-375 and RNU48 as an endogenous control (Applied Biosystems). An ABI Prism 7500 Fast Real-Time PCR System (Applied Biosystems) was used for quantitative real-time PCR analysis using 20× probes for hsa-miR-375 and RNU48. The expression levels of miR-375 were normalized to levels of RNU48. Technical triplicates were performed.

### Wound Healing Assay

Six-well plates were seeded with 1.2 million cells per well and transfected using the precursor molecules as above. Forty-eight hours after transfection, the wells were scratched lightly with 200 μl pipette tips. The rate of wound closure was recorded at 12-hour intervals for 60 hours using 20× magnified microscope photographs. Wound closure was then measured using a fixed angle ruler tool in Adobe Photoshop CS3. Experimental and control conditions were performed in duplicate.

### Proliferation Assay

96-well plates were seeded with 5000 cells per well. Cells were stained with 20 μL per well of CellTiter 96 Aqueous One Solution (Promega) and incubated for one hour at 37° C. before plates were read using a SpectraMax M2 Microtiter Plate Reader. Plates were then analyzed at 6 hours to establish a baseline reading, followed by readings at 24, 48, and 72 hours. SoftMax Pro software was used to record absorbance at the indicated time points. Experimental and control conditions were performed in 12 technical replicates.

### Clonogenic Assay and CDDP Treatment

For clonogenic survival studies, 10 mm dishes were seeded with 3000 transfected cells and either allowed to grow for 14 days to examine basal growth of transfected cells or allowed to adhere overnight before treating with 0.5 μM cisplatin (CDDP) or dimethyl formamide (DMF) control for 24 hrs before allowing to grow for 14 days. Experimental and control conditions were performed in triplicate.

### Results

Transfection of Cells with miR-375 miRNA Mimic Leads to Strong Overexpression of miR-375, Increased Colony Growth and Migration

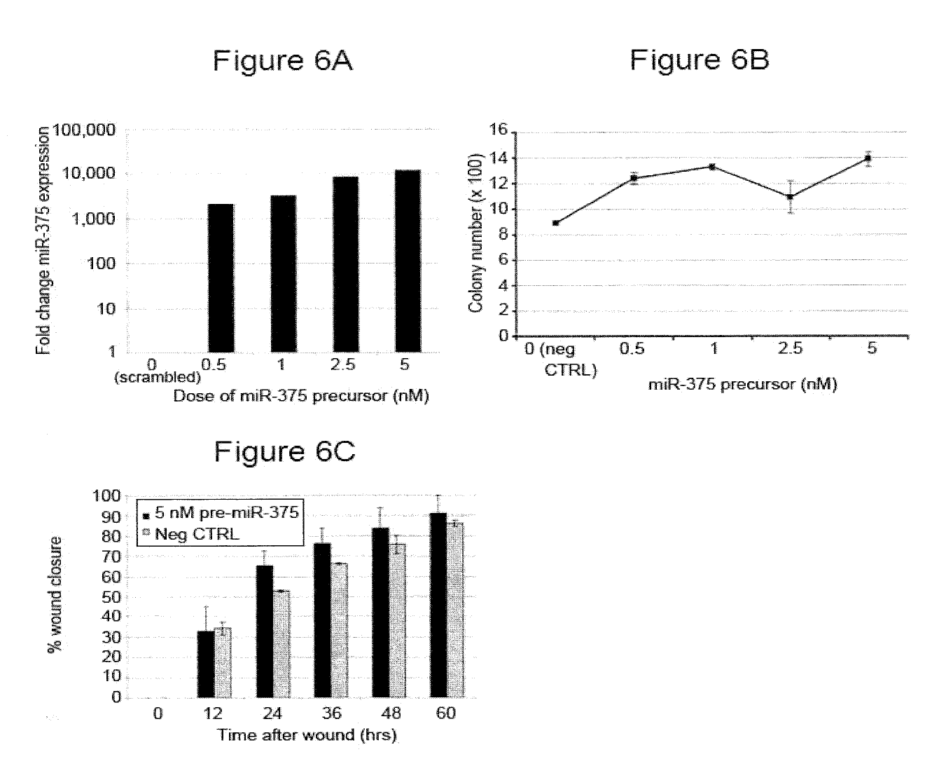

Precursor molecules mimicking the mature form of miR-375 or a control sequence with no known gene target were transfected into the invasive head and neck squamous cell carcinoma cell line, FaDu, at increasing concentrations. Real-time PCR analysis showed strong upregulation of miR-375, from 2000 fold to >10,000 fold overexpression compared to control-transfected cells (FIG. 6A).

Effects of increasing miR-375 expression on FaDu cells were assessed by clonogenic assay, which showed an overall increase in colony formation across transfection groups (FIG. 6B; P=0.002, ANOVA). A wound healing assay was performed following transfection with 5 nM miR-375 precursor or negative control. miR-375-transfected cells tended to show faster wound closure compared to control (FIG. 6C).

Transfection of Cells with miR-375 miRNA Mimic Leads to Increased Proliferation and Resistance to CDDP

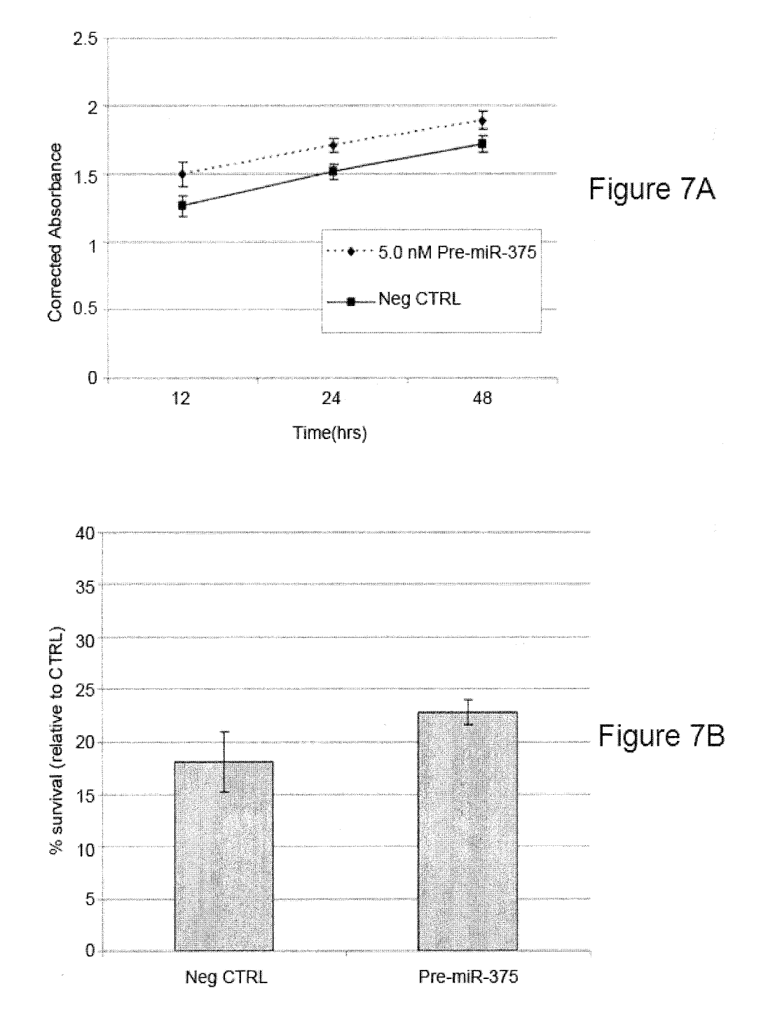

A cell proliferation assay was used to test the effects of miR-375 mimic-transfection on proliferation of FaDu cells. Results showed that miR-375-transfected cells proliferated slightly faster than control-transfected cells (FIG. 7A). To test the effect of miR-375 mimic-transfection on sensitivity to CDDP, a commonly used chemotherapeutic drug used in the treatment of head and neck squamous cell carcinoma (11), a clonogenic assay was performed following 0.5 μM CDDP exposure. miR-375-transfected cells showed greater survival following CDDP treatment than negative control-transfected cells (FIG. 7B).

Transient Transfection of Cells with miR-375 Expression Constructs Leads to Modest Overexpression of miR-375 Decreased Proliferation and Increased Sensitivity to CDDP

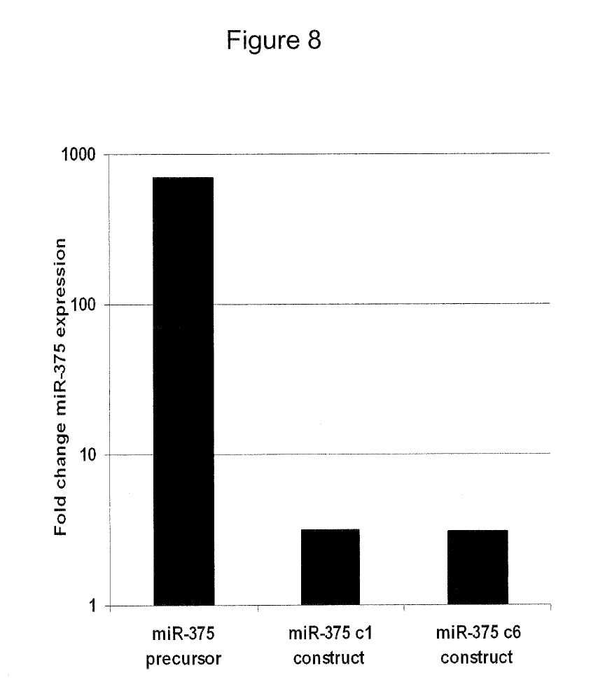

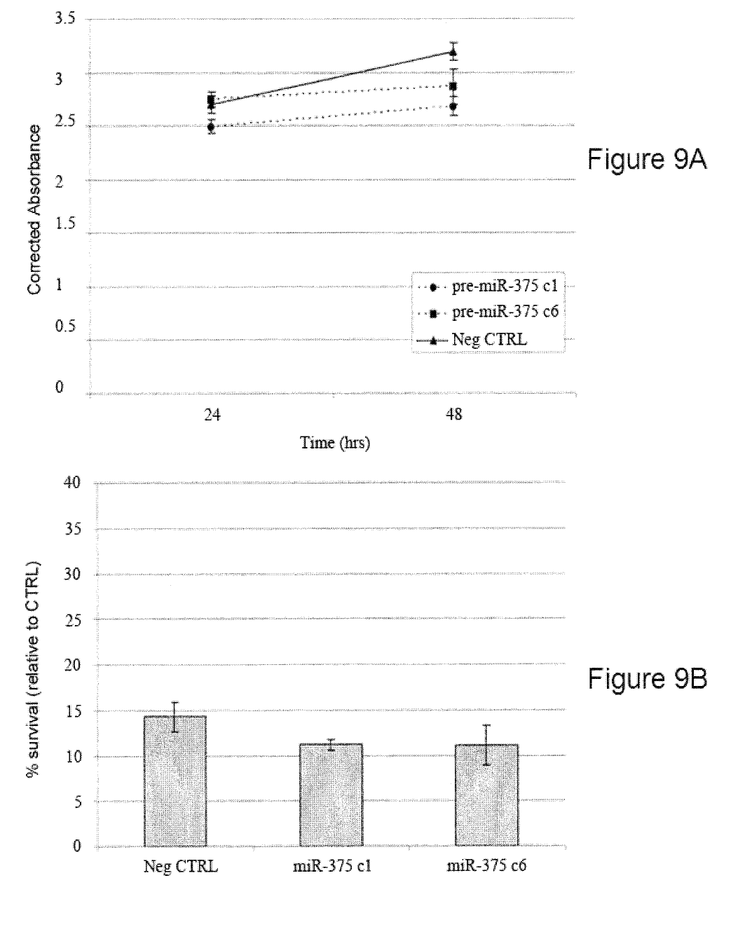

miR-375 expression constructs were created by inserting the premature miR-375 sequence into a Pol II expression vector and two transformants were selected for purification. Transient transfection efficiency was determined to be ˜40%, determined by counting the GFP-positive cells (data not shown). Following transfection of expression plasmids into FaDu cells, miR-375 expression was assessed using real-time PCR. miR-375 expression in both constructs showed modest overexpression (−4 fold) compared to cells transfected with even a low concentration (0.5 nM) of miR-375 precursor molecules (FIG. 8). Contrary to the results seen following transfection with the miRNA mimic, cells transfected with miR-375 expression constructs exhibited slower rates of proliferation and greater sensitivity to CDDP compared to negative control-transfected cells (FIG. 9A-B).

### Discussion

In vitro miRNA gain-of-function studies are important for identifying targets and determining biological function. This work has demonstrated two different methods of overexpressing miR-375, a miRNA that was found to be down-regulated in head and neck squamous cell carcinoma. miR-375 may act as a tumor-suppressor miRNA, normally targeting genes that are upregulated in cancer, such as its known target, PDPK-1 (6). Increasing miR-375 expression in cancer cells may rescue some of their malignant characteristics, such as increased proliferation, migration and resistance to chemotherapeutic agents. Though both types of transfections tested are commonly used in overexpression studies of miRNA, they produce drastically different results, both in the level of upregulation of the miRNA and the resultant phenotype seen in transfected cells.

The first method used was the more simplistic of the two, involving transfection, via a lipid-based transfection agent, of a small double stranded miRNA molecule, analogous to the miRNA in its mature form. The duplex is chemically modified in such a way as to ensure strand specificity and the details of this modification are likely based on the current comprehension of strand bias in small RNAs. In siRNA, the strand incorporated into RISC is the one with the 5′ end that is less tightly paired to its complement (12). In human miRNA, it is known that the active strand shows U enrichment while the alternate strand shows C enrichment at its 5′ end (13). Additionally the purine/pyrimidine content of the two strands is markedly different. Once inside cells, the active strand, having an identical sequence to the mature miRNA of interest, binds the RISC in the cytoplasm and carries out its function of targeting its cognate mRNAs.

The second transfection method involved the generation of a miR-375 expression vector which contains the premature sequence of miR-375 under the control of a constitutive CMV immediate early promoter. Upon transfection, the plasmid enters the nucleus, the promoter is recognized by Pol II, and the pre-miR-375 sequence is transcribed. From this point, the premature sequence likely forms a hairpin structure and is exported and further processed alongside endogenous pre-miRNAs.

Overexpression of miR-375 using the miRNA precursor molecule system resulted in massive upregulation of the mature miRNA, increases in colony formation, proliferation, and migration, as measured by wound healing assay. Consistent with this, miR-375 mimic-transfected cells were also more resistant CDDP, a chemotherapeutic agent commonly used to treat head and neck squamous cell carcinoma (11). These results seemed to oppose the belief that miR-375 acts as a tumor-suppressor miRNA. However, the extremely high level of miR-375 upregulation seen when using this transfection method calls into question the reliability of the results.

Previous work showed that in primary tissues, there was a 22-fold reduction in miR-375 expression in head and neck squamous cell carcinoma tumors compared to normal epithelia (5). Endogenous miRNAs are a tightly regulated class of molecules which, in their mature form, are quite stable (14). Due to their wide regulatory scope, even small changes in miRNA expression, on the order of 2 or 3 fold-change, have been shown to influence biological function significantly (15,16). In the precursor transfection system, a large number of these mature miRNA analogs are transfected into cells and function at the level of the mature miRNA. It is possible that oversaturation of the enzymatic effector complex, RISC, with these more stable, nonendogenous mimics may be interfering with the targeting functions of the many other miRNA that depend on RISC, resulting in an artifactual result related to inhibition of the functional pathway.

Using the expression vector system to increase miR-375 levels resulted in a much more modest ˜4 fold upregulation of the miRNA. Experiments also showed results opposite to the previous system, including slower proliferation and increased sensitivity to CDDP in miR-375-transfected cells compared to control. This system requires far more steps and is more complex than the previous system, as plasmid DNA must enter the nucleus and be recognized, transcribed and processed by nuclear enzymes. This is likely to result in far fewer mature miRNA being generated, as the processing rate is limited by endogenous cell machinery. Additionally, since a transient transfection only results in ˜40% transfection efficiency, the population of cells in which miRNA expression is measured is quite heterogeneous, contributing to a lower expression change and a more muted phenotypic change when examining an entire population of cells.

Several experiments could be performed to confirm or address the potential problems inherent in the systems used to overexpress miR-375. A RISC activity assay, as described by Liu et al. (17), can be performed to confirm the that miRNA mimics overwhelm the RISC. Additionally, the miRNA/RISC complexes can be isolated to determine the proportion of functional miR-375 vs other miRNAs bound to RISC (18). Lysates of transfected cells would be subjected to immunoprecipitation using antibodies against Ago-2 proteins which interact with miRNAs in the RISC.

The expression vector system allows for generation of a stable line of cells overexpressing miR-375. Stable transfection may yield a more homogeneous population of cells with higher expression of miR-375 and a more dramatic phenotypic change. Though this may have the desired effect on miR-375 level, the observed phenotype may be influenced by compensatory mechanisms exerted by the cells in response to the vector's integration into the genome and the resultant constitutive expression of the miRNA.

The use of a luciferase reporter assay based on the mir-375 target sequence would help to elucidate the relative levels of miR-375 function in the two systems used (19). A construct bearing the firefly luciferase mRNA with the 3′ UTR sequence of a known target of miR-375, PDPK-1, or a perfect complement of miR-375 could be designed and co-transfected with either the miR-375 mimic or expression construct and luciferase activity could be assayed to determine whether the transfected miRNAs are functioning as they do endogenously, inhibiting their target mRNA.

Compared to the miRNA precursor system, the expression vector system for miRNA overexpression seems to be a more relevant method for increasing miRNA expression as it more closely mirrors true miRNA processing and results in expression changes on the order of magnitude of physiologic miRNA alterations. To determine which system is best suited for miRNA functional analysis, additional work must be done to determine which allows for proper miRNA function without disruption of endogenous miRNA machinery.

### REFERENCES

1. Ambros, V. (2004) The functions of animal microRNAs. Nature, 431, 350-5.
2. Filipowicz, W., Bhattacharyya, S. N. and Sonenberg, N. (2008) Mechanisms of post-transcriptional regulation by microRNAs: are the answers in sight? Nat Rev Genet, 9, 102-14.
3. Croce, C. M. (2009) Causes and consequences of microRNA dysregulation in cancer. Nat Rev Genet, 10, 704-14.
4. Poy, M. N., Hausser, J., Trajkovski, M., Braun, M., Collins, S., Rorsman, P., Zavolan, M. and Stoffel, M. (2009) miR-375 maintains normal pancreatic alpha- and beta-cell mass. Proc Natl Acad Sci USA, 106, 5813-8.
5. Avissar, M., Christensen, B. C., Kelsey, K. T. and Marsit, C. J. (2009) MicroRNA Expression Ratio Is Predictive of Head and Neck Squamous Cell Carcinoma. Clin Cancer Res, 15, 2850-5.
6. El Ouaamari, A., Baroukh, N., Martens, G. A., Lebrun, P., Pipeleers, D. and van Obberghen, E. (2008) miR-375 targets 3′-phosphoinositide-dependent protein kinase-1 and regulates glucose-induced biological responses in pancreatic beta-cells. Diabetes, 57, 2708-17.
Mora, A., Komander, D., van Aalten, D. M, and Alessi, D. R. (2004) PDK1, the master regulator of AGC kinase signal transduction. Semin Cell Dev Biol, 15, 161-70.
8. Schmittgen, T. D. (2008) Part B—microRNAs: microRNA methods. Methods, 44, 1-2.
9. Blenkiron, C., Goldstein, L. D., Thorne, N. P., Spiteri, I., Chin, S. F., Dunning, M. J., Barbosa-Morais, N. L., Teschendorff, A. E., Green, A. R., Ellis, I. O., Tavare, S., Caldas, C. and Miska, E. A. (2007) MicroRNA expression profiling of human breast cancer identifies new markers of tumor subtype. Genome Biol, 8, R214.
10. Szafranska, A. E., Davison, T. S., John, J., Cannon, T., Sipos, B., Maghnouj, A., Labourier, E. and Hahn, S. A. (2007) MicroRNA expression alterations are linked to tumorigenesis and non-neoplastic processes in pancreatic ductal adenocarcinoma. Oncogene, 26, 4442-52.
11. Matta, A. and Ralhan, R. (2009) Overview of current and future biologically based targeted therapies in head and neck squamous cell carcinoma. Head Neck Oncol, 1, 6.
12. Schwarz, D. S., Hutvagner, G., Du, T., Xu, Z., Aronin, N. and Zamore, P. D. (2003) Asymmetry in the assembly of the RNAi enzyme complex. Cell, 115, 199-208.
13. Hu, H. Y., Yan, Z., Xu, Y., Hu, H., Menzel, C., Zhou, Y. H., Chen, W. and Khaitovich, P. (2009) Sequence features associated with microRNA strand selection in humans and flies. BMC Genomics, 10, 413.
14. Schmittgen, T. D., Lee, E. J., Jiang, J., Sarkar, A., Yang, L., Elton, T. S, and Chen, C. (2008) Real-time PCR quantification of precursor and mature microRNA. Methods, 44, 31-8.
15. Calin, G. A. and Croce, C. M. (2006) MicroRNA-cancer connection: the beginning of a new tale. Cancer Res, 66, 7390-4.
16. Peltier, H. J. and Latham, G. J. (2008) Normalization of microRNA expression levels in quantitative RT-PCR assays: identification of suitable reference RNA targets in normal and cancerous human solid tissues. RNA, 14, 844-52.
17. Liu, Q., Rand, T. A., Kalidas, S., Du, F., Kim, H. E., Smith, D. P. and Wang, X. (2003) R2D2, a bridge between the initiation and effector steps of the Drosophila RNAi pathway. Science, 301, 1921-5.
18. Goff, L. A., Davila, J., Swerdel, M. R., Moore, J. C., Cohen, R. I., Wu, H., Sun, Y. E, and Hart, R. P. (2009) Ago2 immunoprecipitation identifies predicted microRNAs in human embryonic stem cells and neural precursors. PLoS One, 4, e7192.
19. Gibcus, J. H., Tan, L. P., Harms, G., Schakel, R. N., de Jong, D., Blokzijl, T., Moller, P., Poppema, S., Kroesen, B. J. and van den Berg, A. (2009) Hodgkin lymphoma cell lines are characterized by a specific miRNA expression profile. Neoplasia, 11, 167-76.

The teachings of all of the references cited herein are hereby incorporated by reference in their entirety.

### EQUIVALENTS

While this invention has been particularly shown and described with references to example embodiments thereof, it will be understood by those skilled in the art that various changes in form and details may be made therein without departing from the scope of the invention encompassed by the appended claims.

What is claimed is:

1. A method of diagnosing a carcinoma, comprising the step of determining at least one expression ratio selected from the group consisting of a miR-21/miR-375 expression ratio, a miR-181d/miR-375 expression ratio, a miR-181b/miR-375 expression ratio, a miR-491/miR-375 expression ratio, a miR-455/miR-375 expression ratio, a miR-18a/miR-375 expression ratio, a miR-130b/miR-375 expression ratio, a miR-221/miR-375 expression ratio, a miR-193b/miR-375 expression ratio, a miR-181a/miR-375 expression ratio, and a miR-18b/miR-375 expression ratio in a sample, wherein a ratio greater than about 1.0 is diagnostic of the carcinoma and identifies a subject that would potentially benefit from a therapy to treat the carcinoma.

2. The method of claim 1, wherein the expression ratio is the miR-221/miR-375 expression ratio.

3. The method of claim 1, wherein the expression ratio is the miR-21/miR-375 expression ratio.

4. The method of claim 1, wherein the expression ratio is the miR-18a/miR-375 expression ratio.

5. The method of claim 1, wherein the carcinoma is a squamous cell carcinoma.

6. The method of claim 5, wherein the squamous cell carcinoma is at least one member selected from the group consisting of a head squamous cell carcinoma and a neck squamous cell carcinoma.

7. The method of claim 6, wherein the head squamous cell carcinoma is an oral cavity squamous cell carcinoma.

8. The method of claim 6, wherein the neck squamous cell carcinoma includes at least one member selected from the group consisting of a pharyngeal squamous cell carcinoma and a laryngeal squamous cell carcinoma.

9. The method of claim 1, wherein the carcinoma is an adenocarcinoma.

10. The method of claim 9, wherein the adenocarcinoma is an esophageal adenocarcinoma.

11. The method of claim 1, wherein the sample includes a tissue sample.

12. The method of claim 11, wherein the tissue sample includes at least one member selected from the group consisting of an esophageal tissue sample, a laryngeal tissue sample, a pharyngeal tissue sample and an oral tissue sample.

13. The method of claim 11, wherein the tissue sample includes at least a portion of a tumor.

14. The method of claim 13, wherein the tumor is an early-stage tumor.

15. The method of claim 13, wherein the tumor is a human papilloma virus positive tumor.

16. The method of claim 13, wherein the tumor is a human papilloma virus negative tumor.

17. The method of claim 11, wherein the tissue sample includes a preneoplastic lesion.

18. The method of claim 1, wherein the sample includes a saliva sample.

19. A method of diagnosing a carcinoma, comprising the step of comparing an expression level of at least two microRNAs selected from the group consisting of miR-21, miR-181d, miR-181b, miR-491, miR-455, miR-18a, miR-130b, miR-221, miR-193b, miR-181a, miR-18b and miR-375 in a sample from the subject to a corresponding control expression level, wherein a difference in the expression level of the microRNAs in the sample relative to the control expression level is diagnostic of the carcinoma and identifies a subject that would potentially benefit from a therapy to treat the carcinoma.

20. The method of claim 19, wherein the miR-375 expression level in the sample is less than the control miR-375 control expression level.

21. The method of claim 19, wherein the carcinoma is a squamous cell carcinoma.

22. The method of claim 19, wherein the squamous cell carcinoma is at least one member selected from the group consisting of a head squamous cell carcinoma and a neck squamous cell carcinoma.

23. The method of claim 21, wherein the squamous cell carcinoma is at least one member selected from the group consisting of an oral cavity squamous cell carcinoma, a pharyngeal squamous cell carcinoma and a laryngeal squamous cell carcinoma.

24. The method of claim 1, wherein the carcinoma is an adenocarcinoma.

25. The method of claim 24, wherein the adenocarcinoma is an esophageal adenocarcinoma.

26. The method of claim 19, wherein the sample includes a tissue sample.

27. The method of claim 26, wherein the tissue includes at least one member selected from the group consisting of an esophageal tissue sample, a laryngeal tissue sample, a pharyngeal tissue sample and an oral tissue sample.

28. The method of claim 26, wherein the tissue sample includes at least a portion of a tumor.

29. The method of claim 28, wherein the tumor is an early-stage tumor.

30. The method of claim 28, wherein the tumor is a human papilloma virus positive tumor.

31. The method of claim 28, wherein the tumor is a human papilloma virus negative tumor.

32. The method of claim 26, wherein the tissue sample includes a preneoplastic lesion.

33. The method of claim 17, wherein the sample includes a saliva sample.

34. A method of diagnosing a carcinoma selected from the group consisting of a head squamous cell carcinoma and a neck squamous cell carcinoma, comprising the step of comparing an expression level of at least one microRNA selected from the group consisting of miR-181d, miR-181b, miR-491, miR-455, miR-18a, miR-130b, miR-221, miR-193b, miR-181a, miR-18b and miR-375 in a sample from the subject to a corresponding control expression level, wherein a difference in the expression level of the microRNA in the sample relative to the control expression level is diagnostic of the head squamous cell carcinoma and the neck squamous cell carcinoma and identifies a subject that would potentially benefit from a therapy to treat the head squamous cell carcinoma and the neck squamous cell carcinoma.

35. The method of claim 34, wherein the squamous cell carcinoma is the head squamous cell carcinoma.

36. The method of claim 34, wherein the squamous cell carcinoma is the neck squamous cell carcinoma.

37. The method of claim 34, wherein the expression level of the microRNA in the sample is at least about two-fold greater than the control expression level.

38. The method of claim 34, wherein the expression level of the microRNA in the sample is at least about twenty-fold less than the control expression level.

39. The method of claim 34, further comprising the step of comparing the expression level of miR-21 in the sample to a miR-21 control expression level.

40. The method of claim 34, wherein the sample includes a tissue sample.

41. The method of claim 40, wherein the tissue sample is at least one member selected from the group consisting of an esophageal tissue sample, a laryngeal tissue sample, a pharyngeal tissue sample and an oral tissue sample.

42. The method of claim 40, wherein the tissue sample includes at least a portion of a tumor.

43. The method of claim 42, wherein the tumor is an early-stage tumor.

44. The method of claim 42, wherein the tumor is a human papilloma virus positive tumor.

45. The method of claim 42, wherein the tumor is a human papilloma virus negative tumor.

46. The method of claim 40, wherein the tissue sample includes a preneoplastic lesion.

47. The method of claim 34, wherein the sample includes a saliva sample.

48. A method of treating a squamous cell carcinoma in a subject, comprising the step of administering a nucleic acid encoding a miR-375 gene product to the subject.

49. The method of claim 48, wherein the squamous cell carcinoma is selected from the group consisting of a head squamous cell carcinoma and a neck squamous cell carcinoma.

50. The method of claim 48, wherein the nucleic acid encoding the miR-375 gene product includes an expression vector.

51. The method of claim 50, wherein the expression vector includes an RNA polymerase II promoter.

52. The method of claim 51, wherein the RNA polymerase II promoter includes a constitutive promoter.

53. The method of claim 52, wherein the constitutive promoter includes a human cytomegalovirus immediate early promoter.

54. The method of claim 48, wherein the nucleic acid encoding the miR-375 gene product includes a nucleic acid that encodes a premature miR-375 gene product.

55. A method of optimizing treatment of a subject having a squamous cell carcinoma, comprising the step of determining an expression level of a miR-21 gene product in a sample from the subject, wherein overexpression level of the miR-21 gene product in the sample compared to expression in a reference miR21-gene product identifies a subject that has an aggressive squamous cell carcinoma that would potentially benefit from a therapy to treat the aggressive squamous cell carcinoma.

56. The method of claim 55, wherein the squamous cell carcinoma is selected from the group consisting of a head squamous cell carcinoma and a neck squamous cell carcinoma.
